(19) United States
(12) Patent Application Publication
(10) Pub. No.: US 2025/0352804 A1
(43) Pub. Date: Nov. 20, 2025

(54) DETACHABLE AND REVERSIBLE MATTRESS TICKING ASSEMBLIES AND MATTRESSES EMPLOYING THE SAME

(71) Applicant: kickball concepts llc, Sterling, VA (US)

(72) Inventors: Harrison Robert MURPHY, St. Petersburg, FL (US); Juraj Michal Daniel SLAVIK, II, McLean, VA (US)

(73) Assignee: kickball concepts llc, Sterling, VA (US)

(21) Appl. No.: 19/282,590

(22) Filed: Jul. 28, 2025

Related U.S. Application Data
(63) Continuation of application No. 19/078,879, filed on Mar. 13, 2025.
(60) Provisional application No. 62/926,075, filed on Oct. 25, 2019.

(51) Int. Cl. A61N 1/365; A61B 5/283; A61B 5/316; A61B 18/00; A61B 18/12; A61N 1/05; A61N 1/32

(52) U.S. Cl. CPC A61N 1/36507; A61B 5/283; A61B 5/316; A61N 1/056; A61B 2018/00351; A61B 2018/00613; A61B 2018/00839; A61B 18/12; A61N 1/327

(57) ABSTRACT

Detachable and reversible mattress ticking assembly may be constructed so as to provide consumers the ability self-modify the orientation of the reversible mattress ticking so as to enjoy dissimilar performance attributes that the reversible ticking assembly or to merely extend the useful service life of the mattress ticking assembly when one side or the other becomes worn or aesthetically diminished.

### CLAIM TO PRIORITY APPLICATIONS

This application is a continuation application of U.S. patent application Ser. No. 17/078,879, filed Oct. 23, 2020, entitled “Detachable And Reversible Mattress Ticking Assemblies And Mattresses Employing The Same” (atty. dock. no. 3252.009A), which application claims the benefit of U.S. Provisional Application No. 62/926,075, filed Oct. 25, 2019, entitled “Detachable And Reversible Mattress Ticking Assemblies And Mattresses Employing The Same” (atty. dock. no. 3252.009P1), which applications are hereby incorporated herein by reference in their entireties.

### TECHNICAL FIELD

The present disclosure relates generally to mattresses that possess design enhancements anticipated to be both desirable to modern consumers, including a detachable and reversible mattress ticking assembly that is capable of delivering dissimilar or interchangeable performance attributes and may optionally incorporate design attributes that facilitate consumer access to the inner filling materials of the mattresses.

### BACKGROUND

Conventional tape-edged, inner spring mattresses employ a ticking that surrounds a core, e.g., a core of supportive filling materials is encased in an outermost layer of fabric or related material that is referred to as the ticking. The ticking has historically been permanently installed on the mattress by means of sewn seams, commonly referred to as the “taped edge”, used at all points that separate pieces of fabric are cut into patterns to form the ticking and by the fastening of ticking fabric elements to the innerspring construction by means of metal hog-rings. The ticking is completely sewn shut around the mattress core. The mattress as a unit may be repositioned, e.g., rotated and/or flipped relative to a mattress foundation, by a user. A user is not able to remove the sewn ticking from the core of the mattress.

Recently, mattresses are being sold direct-to-customer (DTC), e.g., mattresses sold as “bed-in-a-box.” Typically, such mattresses include a foam core and a cover with a ticking disposed on the outside of the cover. The cover includes various fabrics with sewn seams and a zipper to allow a consumer to remove the cover, for example, for cleaning. The zipper has a usable tab on the ticking side of the cover to allow the consumer to dispose the cover around the core with the ticking disposed on the outside of the cover.

### SUMMARY

Shortcomings of the prior art are overcome, and additional advantages are provided through the provision of a mattress assembly which includes, a mattress core having a top side, a bottom side, and a peripherally extending side disposed between the top side and the bottom side, and a detachable and reversible mattress ticking assembly for at least partially enclosing the mattress core. The detachable and reversible mattress ticking assembly may include a single textile or a plurality of textiles joined together defining an obverse face and a reverse face of the detachable and reversible mattress ticking assembly. The detachable and reversible mattress ticking assembly is disposable in a first orientation with the reverse face facing the mattress core and the obverse face facing outwardly defining a ticking surface. The detachable and reversible mattress ticking assembly is disposable in a second orientation with the obverse face facing the mattress core and the reverse face facing outwardly defining a ticking surface. The mattress assembly satisfies the flammability standards of both 16 CFR 1632.4 and 16 CFR 1633 when tested with the detachable and reversible mattress ticking assembly disposed and secured in the first orientation, and the mattress assembly satisfies the flammability standards of both 16 CFR 1632.4 and 16 CFR 1633 when tested with the detachable and reversible mattress ticking assembly disposed and secured in the second orientation.

In another embodiment, a method forming a mattress assembly, may include, for example, providing a mattress core having a top side, a bottom side, and a peripherally-extending side disposed between the top side and the bottom side, positioning a detachable and reversible mattress ticking assembly over the mattress core in a first orientation, and wherein the mattress assembly meets the flammability standards of both 16 CFR 1632.4 and 16 CFR 1633 when tested with the detachable and reversible mattress ticking assembly disposed in the first orientation covering the mattress core with the reverse face facing the mattress core and the obverse face facing outwardly from the mattress assembly, and wherein the mattress assembly meets the flammability standards of both 16 CFR 1632.4 and 16 CFR 1633 when tested with the detachable and reversible mattress ticking assembly disposed in a second orientation covering the mattress core with the obverse face facing the mattress core and the reverse face facing outwardly from the mattress assembly. The method may further include removing the detachable and reversible mattress ticking assembly from the mattress core, and repositioning the detachable and reversible mattress ticking assembly over the mattress core in the second orientation.

In another embodiment, a mattress foundation, may include, for example, a core having a top side, a bottom side, and a peripherally extending side disposed between the top side and the bottom side, and a detachable and reversible ticking assembly for at least partially enclosing the core. The detachable and reversible ticking assembly may include a single textile or a plurality of textiles joined together having an obverse face and a reverse face. The detachable and reversible mattress ticking assembly disposable in a first orientation with the reverse face facing the core and the obverse face facing outwardly, and in a second orientation with the obverse face facing the core and the reverse face facing outwardly. The mattress foundation satisfies the flammability standards of both 16 CFR 1632.4 and 16 CFR 1633 when tested with the detachable and reversible ticking assembly is disposed and secured in the first orientation, and the mattress foundation satisfies the flammability standards of both 16 CFR 1632.4 and 16 CFR 1633 when tested with the detachable and reversible ticking assembly is disposed and secured in the second orientation.

In another embodiment, the mattress assemblies may include, for example, a mattress core assembly of supportive filling materials and a detachable and reversible mattress ticking assembly for covering the mattress core assembly. The mattress core assembly may include top side, a bottom side, and peripherally extending sides disposed between the top side and the bottom side. The mattress core assembly may include a single, homogenous supportive material or a potentially unlimited combination of similar and/or dissimilar filling materials. The mattress assembly described herein may be specifically designed to meet the flammability standards of 16 CFR 1633 and 16 CFR 1632 when the detachable and reversible mattress ticking assembly is installed over the mattress core assembly in either an obverse orientation or a reverse orientation.

The present disclosure may also have applicability outside of the United States and it is anticipated that the design flexibility provided may enable the mattress using this approach to meet flammability in jurisdictions outside of the USA, such as requirements in the United Kingdom of cigarette ignition sources under EN 597-2 and open flame sources under EN 597-2. The design approach could also be enhanced to enable compliance with a Crib 5 level ignition source under BS 6807 Chapter 9. Similarly, furniture articles using this approach may be able to meet cigarette ignition sources under EN 1021-1 and open flame ignition sources under 1021-2. Enhanced designs could also be developed to meet the Crib 5 level ignition source under BS 5852 Chapters 11 or 12. It is contemplated that this present disclosure may possess the capability of complying with similar requirements of other countries as well.

The particular flammability test protocols identified herein are incorporated in their entirety by reference.

In another embodiment, the mattress assemblies may include, for example, a mattress core assembly of supportive filling materials having a top side, a bottom side, and peripherally extending sides disposed between the top side and the bottom side, where the mattress assembly is at least partially enclosed in a textile encasement and then a detachable and reversible mattress ticking assembly having a top side extending over the top side of the mattress core assembly and a peripheral portion extending over the peripherally extending sides of the mattress core assembly, and a bottom side disposed under a portion of the bottom side of the mattress core, and wherein the mattress assembly meets the flammability standards of the 16 CFR 1633 when the detachable and reversible mattress ticking assembly is installed over the mattress core assembly in either possible orientation.

Still another embodiment may deliver the benefit of the contemplated reversible nature of the present disclosure but only a portion of the mattress ticking assembly may be reversible. In this embodiment, the bottom side and the peripherally extending sides disposed between the top side and the bottom side may be designed to not be reversible and it may be only the top side of the ticking assembly that is reversible.

At one fundamental level, the reversibility capability of the present disclosure may provide multiple possible configuration variations not presently provided for or contemplated by the prior art or industry practice.

A first configuration variation of the reversibility and detachable mattress ticking assembly may provide for an obverse face and a reverse face that are aesthetically identical. This variation may be primarily limited to promoting extended service life for the mattress since the user can simply reverse the ticking assembly when one face shows wear or the effects of soiling that cannot be washed out of the detachable ticking assembly.

A second configuration variation may provide for an obverse face and a reverse face that are aesthetically different. The differences may be color, pattern, texture or textile construction of the textile or ticking used for the two faces-typically differences that are readily apparent from only visual examination. This variation may convey the same benefits as the first configuration variation as well as the added benefit of providing the ability to choose alternate visual appearances of the mattress while in use over time.

A third configuration variation may provide for an obverse face and a reverse face that possess similar performance attributes. Certain textile products can be engineered to deliver performance-oriented benefits, such as cooling properties, warming properties, therapeutic properties and the like. Such properties, which are increasingly in demand from consumers, may be part of the inherent material formulation or the result of treatments or finishes applied to the fibers, yarns or fabrics used as ticking fabrics. The service life expectancy of such performance features may be less than permanent and may diminish over time. In the configuration variation that disposes the same performance-oriented benefit on both faces of the detachable and reversible mattress ticking assembly of the current disclosure, the potential is provided that could as much as double the service life expectancy of the benefits in the consumers' original purchase.

A fourth configuration variation may be similar to the third listed above, but this may provide for embodiments that offer dissimilar performance attributes on the obverse and reverse faces. For instance, an obverse that heats and a reverse that cools.

A number of further configuration variations may also possible that combine the preceding four examples. For instance, the second variation of an aesthetic, such as color, could be combined with the fourth configuration variation of dissimilar performance attributes. An example may have the obverse face be a blue color with cooling performance properties and the reverse face be a red color with heating performance properties.

Each of the foregoing contemplated configuration variations may be reasonably expected to be able to be created using either a design approach where the ticking assembly is a design that fully encloses the mattress core assembly on the entirety of all six planar faces or as a design that encloses the mattress core assembly on the entirety of the planar face intended to serve as the top panel of mattress (closest to the mattress user), the four peripherally attached vertical faces and at least a portion of the bottom planar face.

The possible embodiments of the disclosure disclosed herein may have the intended flexibility to impart the reversibility attributes to all surface faces of the ticking assembly or to only a portion of the surface faces that may help reduce material and design costs while not materially reducing or limiting the benefits to be realized from the approach.

The present disclosure portends to deliver design flexibility to manufacturers and product choices to consumers that heretofore have not been provided by the prior art or industry practice.

### BRIEF DESCRIPTION OF THE DRAWINGS

The subject matter which is regarded as the disclosure is particularly pointed out and distinctly claimed in the concluding portion of the specification. The disclosure, however, may best be understood by reference to the following detailed description of various embodiments and the accompanying drawings in which:

FIG. 1 is a perspective view of a mattress assembly and a foundation according to an embodiment of the present disclosure.

FIG. 2 is an enlarged end view of the mattress assembly of FIG. 1.

FIG. 3 is a cross-sectional view taken along line 3-3 of the mattress assembly of FIG. 1 having a core with a single cushioning element at least partially enclosed by an independent fire barrier and then subsequently enclosed by a detachable and reversible mattress ticking assembly.

FIG. 4 is a cross-sectional view of a mattress assembly according to an embodiment of the present disclosure having a core having two cushioning elements at least partially enclosed by an independent fire barrier and then subsequently and at least partially enclosed by a detachable and reversible mattress ticking assembly.

FIG. 5 is a cross-sectional view of a mattress assembly according to an embodiment of the present disclosure having a core with a single cushioning element at least partially enclosed by an independent fire barrier and then subsequently partially enclosed by a detachable and reversible mattress ticking assembly.

FIG. 6 is a cross-sectional view of a mattress assembly according to an embodiment of the present disclosure having a core having multiple cushioning elements at least partially enclosed by an independent fire barrier and then subsequently partially enclosed by a detachable and reversible mattress ticking assembly.

FIG. 7 is a cross-sectional view of a mattress assembly according to an embodiment of the present disclosure having a core with a single cushioning element at least partially enclosed by an independent fire barrier, then encased in a textile cover that is not the outermost fabric of the mattress assembly, and then finally partially enclosed by a detachable and reversible mattress ticking assembly.

FIG. 8 is a cross-sectional view of a mattress assembly according to an embodiment of the present disclosure having a core having multiple cushioning elements then encased in a textile cover that is not the outermost fabric of the mattress assembly and then finally partially enclosed by a detachable and reversible mattress ticking assembly.

FIG. 9 is a perspective view, partially cut away, of a mattress assembly according to an embodiment of the present disclosure having a core assembly of multiple foam layers and a detachable and reversible mattress ticking assembly.

FIG. 10 illustrates a first side and a second side of a two-sided fabric having opposing sides that have dissimilar or similar performance or aesthetic attributes for use in the embodiments of the present disclosure to create a detachable and reversible mattress ticking assembly.

FIG. 11 is a top perspective view of a mattress assembly according to an embodiment of the present disclosure with a detachable and reversible mattress ticking assembly that includes the fabric of FIG. 10 oriented with the first side disposed in an outermost position.

FIG. 12 is a top perspective view of the mattress assembly of FIG. 11 with the detachable and reversible mattress ticking assembly that includes the fabric of FIG. 10 oriented with the second side disposed in an outermost position.

FIG. 13 is a cross-sectional view of a portion of a detachable and reversible mattress ticking assembly according to an embodiment of the present disclosure having a plurality of layers.

FIG. 14 is a cross-sectional view of a portion of a detachable and reversible mattress ticking assembly according to an embodiment of the present disclosure having a plurality of layers.

FIG. 15 is a top perspective view of a mattress assembly according to an embodiment of the present disclosure with a detachable and reversible mattress ticking assembly in a form intended for a double-sided mattress.

FIG. 16 is a top perspective view of a mattress assembly according to an embodiment of the present disclosure with a detachable and reversible mattress ticking assembly extending over a top planar face, and the remainder of the ticking assembly manufactured to not be reversible.

FIG. 17 is a top perspective view of a mattress assembly according to an embodiment of the present disclosure with a detachable and reversible mattress ticking assembly having a partially reversible form where only the top planar face of the mattress ticking assembly is reversible and where the top planar face having different portions.

FIG. 18 is a bottom perspective view of a mattress assembly according to an embodiment of the present disclosure in a form intended for a two-sided mattress.

FIG. 19 is a bottom perspective view of a mattress assembly according to an embodiment of the present disclosure in a form intended for a two-sided mattress.

FIG. 20 is a side elevational view of a zipper assembly according to an embodiment of the present disclosure.

FIG. 21 is a side elevational view of a zipper assembly according to an embodiment of the present disclosure.

FIGS. 22 and 23 are perspective views of a reversible zipper slider head according to an embodiment of the present disclosure having a slider body with a puller that facilitates an end-user selectable reversible characteristics of a detachable and reversible mattress ticking assembly.

FIG. 24 is a perspective view of a reversible zipper slider head according to an embodiment of the present disclosure having two pullers that facilitates the end-user selectable reversible characteristic of a detachable and reversible mattress ticking assembly.

FIG. 25 is a cross-sectional view of a portion of a prior art mattress having a mattress ticking.

FIG. 26 is a cross-sectional view of a portion of a mattress assembly according to an embodiment of the present disclosure having a detachable and reversible ticking assembly having a plurality of flame-retardant flange material disposable around the zipper slider.

FIG. 27 is an elevations side view of a dual side zipper flap or waterfall design according to an embodiment of the present disclosure for use in the detachable and reversible mattress ticking assemblies.

FIG. 28 is a perspective view of a flap or waterfall zipper cover according to an embodiment of the present disclosure for use in the detachable and reversible mattress ticking assemblies.

FIG. 29 is a top view of a mattress assembly according to an embodiment of the present disclosure having a detachable and reversible ticking assembly.

FIG. 30 is a top view of a mattress assembly according to an embodiment of the present disclosure having a detachable and reversible ticking assembly with a zipper assembly operable for detaching and reversing a top panel of the detachable and reversible ticking assembly.

FIG. 31 is a top view of the mattress assembly of FIG. 31 with the top panel of the detachable and reversible ticking assembly reversed.

### DETAILED DESCRIPTION

The present disclosure and certain features, advantages, and details thereof, are explained more fully below with reference to the non-limiting embodiments illustrated in the accompanying drawings. Descriptions of well-known materials, fabrication tools, processing techniques, etc., are omitted so as to not unnecessarily obscure the disclosure in detail. It should be understood, however, that the detailed description and the specific examples, while indicating embodiments of the present disclosure, are given by way of illustration only, and are not by way of limitation. Various substitutions, modifications, additions and/or arrangements within the spirit and/or scope of the underlying concepts will be apparent to those skilled in the art from this disclosure. Reference is made below to the drawings, which are not drawn to scale for ease of understanding, and wherein the same reference numbers used throughout different figures designate the same or similar components.

As described in greater detail below, the technique of the present disclosure may provide detachable and reversible mattress ticking assemblies and mattresses employing the same. The mattresses may have a detachable and reversible mattress ticking assemblies that may afford the end-user with a choice of more than one usable ticking surface or choice of more than one portion of the ticking surface that may surround the core to form the mattress. For example, the mattress assembly may include a detachable and reversible mattress ticking assembly having a first ticking face or surface and a second ticking face or surface. The detachable and reversible mattress ticking assembly may be disposed in a first orientation around the core with the first ticking face or surface facing outwardly and the second ticking face or surface facing the mattress core. The detachable and reversible mattress ticking assembly may be removed from the core, and turned inside out, and repositioned on the core in a second orientation so that the second ticking face or surface faces outwardly and the first ticking face or surface faces the mattress core.

In some embodiments, the one or more choices of the ticking faces or surfaces may be have different colors, different ornamentations, and/or different performance characteristics. In some embodiments, the one or more choices of the ticking face or surfaces may have the same colors, same ornamentations, and/or the same performance characteristics. Such embodiments may extend the life of the mattress for the user, or reposition a soiled ticking assembly and avoid disposing of the entire mattress or the entire ticking assembly itself.

While description and illustration of the present disclosure is made to various embodiments of reversible ticking assemblies for mattresses, the design approaches recited herein may also be found to be beneficial in the production of mattress foundations and articles of upholstered furniture, transportation seating or other similar cushion based articles or furnishing systems where the end-user desires to have the ability to easily access and modify, exchange or replace the internal material elements of such articles or systems and the ability to easily remove and install and even launder a detachable and reversible ticking assembly or exterior most fabric used in the construction of the composite article.

According to the International Sleep Products Association (“ISPA”) 2018 Mattress Industry Trends Report, the entire subject matter of which is incorporated herein by reference, U.S. mattress producers shipped slightly more than 50 million units (mattresses and foundations) in 2018 an increase of 7.4% over 2017. Average unit prices at wholesale declined by 1.7% in 2018.

Mattresses and foundations are typically sold as sets. However, more mattresses are sold annually than foundations. Some mattresses are sold as replacements for existing mattresses (without a new foundation) or are for use in platform beds or other beds that do not require a foundation. ISPA estimated that the total number of U.S. conventional mattress shipments was 28.8 million in 2017, and 30.4 million in 2019. These estimates do not include futons, crib mattresses, juvenile mattresses sleep sofa inserts, or hybrid water mattresses. These “non-conventional” sleep surfaces are estimated to include no more than 10 percent of total annual shipments of all sleep products. The value of conventional mattress and foundation shipments in 2018, according to ISPA, was $7.99 and $1.38 billion respectively, compared to $7.93 and $1.25 billion respectively in 2017.

The expected useful life of mattresses can vary substantially, with more expensive models generally experiencing the longest useful lives. Industry sources have historically recommended replacement of mattresses after 10 to 12 years of service life, however the 2016 Better Sleep Council Consumer Research, the entire subject matter of which is incorporated herein by reference, indicates that consumers expect to replace their mattress every 9.4 years, while in practice the replacement frequency is actually only every 8.9 years. This trend is clear evidence that mattress lives are shortening and as a result the impact of mattress disposal on the waste stream is becoming increased.

ISPA also provides data on the customary sizes of mattresses and the annual sales attributable to each size of mattress. The typical sizes and sales are as follows as show in Table 1 below.

TABLE 1

| | | 2017 | 2018 |
|---|---|---|---|
| Mattress Size | Dimensions | % of Total | % of Total |
| Twin | 38″ × 74.5″ | 17.9% | 17.2% |
| Twin XL | 38″ × 79.5″ | 2.9% | 3.2% |
| Full | 53″ × 74.5″ | 15.0% | 14.8% |
| Full XL | 53″ × 79.5″ | 0.6% | 0.5% |
| Queen | 60″ × 79.5″ | 41.6% | 41.4% |
| King | 76″ × 79.5″ | 17.0% | 17.8% |
| Cal King | 72″ × 84″ | 1.6% | 1.7% |
| Odd Sizes | Varies | 3.4% | 3.4% |

Mattresses have historically been classified into two general types by the International Sleep Products Association (ISPA): Innerspring and Non-Innerspring. ISPA reported the specific sales volumes of each type, however starting in 2015 the ceased this separate reporting. These designs preclude entirely the end-consumers ability to adjust or even replace inner material components of the mattress in the event of material failures, wear or desire to alter the comfort and feel of the mattress.

The last reported data by ISPA that broke out Non-Innerspring and not all of these designs provide the end-user with the ability to access the interior elements of the mattress by means of a ticking assembly that allows opening and closing of the ticking assembly through use of a zipper. These mattresses are typically compressed, sealed in plastic to hold the compression, rolled and then stuffed in packaging that permits the mattresses to be shipped by courier services such as United Parcel Service, Federal Express or DHL. The consumer is responsible for unpackaging the mattress and placing it in their desired sleeping space.

Mattresses are regulated by the Federal Government with regards to their flammability and additionally are subject to numerous state and local regulations with regards to their manufacture, composition and labelling.

The Federal Flammability Act 16 C.F.R. Part 1632 was originally established to provide a federal flammability standard for bedding mattresses to reduce the chance and size of accidental fires caused by a cigarette or some form of smoldering heat source coming into contact with the bedding mattresses. The Consumer Product Safety Commission (“CPSC”) recognized this flammability standard was inadequate due a significant number of mattress fires that were the result of some form of open fuel source.

In October 2001, working in conjunction with the National Institute for Safety and Technology (“NIST”), the CPSC issued an advance notice of proposed rulemaking (“ANPR”) concerning the open flame ignition of mattresses/bedding to broaden the standard to include some form of open fuel source requirement. These flammability standards were researched and in 2005 the CPSC issued a notice of proposed rulemaking (“NPR”) proposing a flammability standard based on the NIST research.

The characteristics of mattress/bedding fires and research conducted to develop the standard are discussed in detail in the NPR, 70 F.R. 2470, incorporated herein by reference in its entirety. The final rule was enacted in March of 2006 as 16 C.F.R. Part 1633 entitled Standard for the Flammability (Open Flame) of Mattress Sets (hereinafter the “1633 flammability standard”) as published in the Federal Register/Vol. 71, No. 50. Mar. 15, 2006/Rules and Regulations incorporated herein by reference in its entirety. The 1633 flammability standard applies to mattresses and mattress and foundation sets (“mattress sets”). A “Mattress” is defined as a resilient material, used alone or in combination with other materials, enclosed in a ticking and intended or promoted for sleeping upon.

In the context of the embodiments of the present disclosure, terms relating to mattresses are defined in conformity with terms as defined by 16 C.F.R. 1632, and as follows:
(a) Mattress means a ticking filled with a resilient material used alone or in combination with other products intended or promoted for sleeping upon.
   (1) This definition includes, but is not limited to, adult mattresses, youth mattresses, crib mattresses including portable crib mattresses, bunk bed mattresses, futons, water beds and air mattresses which contain upholstery material between the ticking and the mattress core, and any detachable mattresses used in any item of upholstered furniture such as convertible sofa bed mattresses, corner group mattresses, day bed mattresses, roll-a-way bed mattresses, high risers, and trundle bed mattresses. See Sec. 1632.8 Glossary of terms, for definitions of these items.
   (2) This definition excludes sleeping bags, pillows, mattress foundations, liquid and gaseous filled tickings such as water beds and air mattresses which do not contain upholstery material between the ticking and the mattress core, upholstered furniture which does not contain a detachable mattress such as chaise lounges, drop-arm love seats, press-back lounges, push-back sofas, sleep lounges, sofa beds (including jackknife sofa beds), sofa lounges (including glide-outs), studio couches and studio divans (including twin studio divans and studio beds), and juvenile product pads such as car bed pads, carriage pads, basket pads, infant carrier and lounge pads, dressing table pads, stroller pads, crib bumpers, and playpen pads. See Sec. 1632.8 Glossary of terms, for definitions of these items.
(b) Mattress Pad means a thin, flat mat or cushion, and/or ticking filled with resilient material for use on top of a mattress. This definition includes, but is not limited to, absorbent mattress pads, flat decubitus pads, and convoluted foam pads which are totally enclosed in ticking. This definition excludes convoluted foam pads which are not totally encased in ticking.
(c) Ticking means the outermost layer of fabric or related material that encloses the core and upholstery materials of a mattress or mattress pad. A mattress ticking may consist of several layers of fabric or related materials quilted together.
(d) Core means the main support system that may be present in a mattress, such as springs, foam, hair block, water bladder, air bladder, or resilient filling.
(e) Upholstery material means all material, either loose or attached, between the mattress or mattress pad ticking and the core of a mattress, if a core is present.
(f) Tape edge (edge) means the seam or border edge of a mattress or mattress pad.
(g) Quilted means stitched with thread or by fusion through the ticking and one or more layers of upholstery material.
(h) Tufted means buttoned or laced through the ticking and upholstery material and/or core, or having the ticking and upholstery material and/or core drawn together at intervals by any other method which produces a series of depressions on the surface.” (16CFR1632.2).
(r) Mattress foundation. Consists of any surface such as foam, box springs or other, upon which a mattress is placed to lend it support for use in sleeping upon.” (16CFR1632.8)

Additionally, in the context of the present disclosure, these terms are further defined in conformity with terms as defined by the Consumer Product Safety Commission, 16 CFR Part 1633, Standard for the Flammability (Open Flame) of Mattress Sets; Final Rule—published in the Federal Register, Mar. 15, 2006.

Another aspect of the present disclosure relates to its use in articles of upholstered furniture. In the context of the present disclosure, terms relating to upholstered furniture are defined in conformity with terms as defined by the draft language of 16 C.F.R. 1634, as published by the CPSC in May 2005, and incorporated in their entirety herein by reference.

Another aspect of the present disclosure relates to its use in other articles filled with resilient cushioning materials. In the context of the present disclosure, terms relating to filled articles and bedding are defined as follows in conformity with the terms defined by the California BHFTI draft of Technical Bulletin #604 published Oct. 1, 2004, and the ANPR for 16 CFR 1634 Standard To Address Open Flame Ignition of Bedclothes published by the CPSC in the Federal Register on Jan. 13, 2005, pages 2514 through 2517, and incorporated in their entirety herein by reference.

Good sleep is widely believed to be critical in enabling people to feel and perform their best, in all aspects of their lives. Sleep is routinely touted by medical professionals as being essential for improved health. Good sleep has been shown to affect numerous aspects of everyday life, from the ability of students to commit new information to memory to preventing weight gain. Because of the myriad of important benefits to be derived from good sleep, it is essential for people to have mattresses and bedding that match their personal sleep preference and are suited to their body type in order for them to enjoy comfortable, restful, beneficial sleep.

The 2012 “Bedroom Poll” conducted by the National Sleep Foundation (NSF), determined that 92% of nearly 1,500 rated that having a comfortable mattress was an important determinant in getting a good night's sleep. Those findings, which can be found at the NSF website, www.sleepfoundation.org, are incorporated herein by reference.

The human body may change over time. Sleeper may lose or gain weight. The change in weight may be slight or significant. When the change in weight is significant, it may affect the type of bedding the sleeper requires to achieve restful sleep. For example, a significant gain in weight may require that a sleeper use a firmer mattress, to provide support for the added weight. Conversely, a material weight loss can similarly affect the consumer's reaction to the comfort and feel of the mattress cushioning elements.

The sleeper's sleep preference may also change over time. For example, a sleeper who typically likes to sleep on their back may, over time, prefer to sleep on his or her side. The change in sleep preference may affect the type of bedding the sleeper requires to achieve restful sleep. For example, a sleeper changing from a back sleeper to a side sleeper may require that the sleeper use a firmer mattress.

In addition to preference changes over long periods of time, there may be seasonal changes that affect a sleeper's ability to achieve restful sleep. Temperature regulation has been known to impact sleep. If a sleeper is too warm or too cold, the nature of their sleep may be adversely affected. Textile technologies have been developed to deliver fabrics that have cooling properties or that have heating properties and the mattress industry has adopted such technologies into ticking fabrics and into foams. The present state of the art only allows consumers to derive the benefit of one approach or the other in the purchase of their mattress and the present disclosure successfully overcomes this deficiency.

The firmness and degree of support provided by a mattress can be influenced by the selection of the cushioning materials used to build a mattress. In the DTC selling model, the innerspring units historically found in most mattresses have been replaced by layers of foam that have been glued together. The most prevalent type of cushioning material used is foam, however foams come in a wide range and variety of compositions and the preparation of the foams can be altered to deliver differing densities as well as differing levels of firmness.

The Polyurethane Foam Association provides a teaching aid for understanding the technical terms related to flexible polyurethane foam (FPF) on their website at http://www.pfa.org/ce/handout2.html and the following definitions are well-known to those skilled in the art. These definitions have also been widely adopted for referring to the physical properties of foams of other compositions including gel foams, synthetic and natural latex foams and blends thereof,

Density—A measurement of the mass per unit volume. It is measured and expressed in pounds per cubic foot (pcf) or kilograms per cubic meter (kg/m3).

Indentation Force Deflection (IFD)—A measure of the load bearing capacity of flexible polyurethane foam. IFD is generally measured as the force (in pounds) required to compress a 50 square inch circular indentor foot into a four inch thick sample, typically 15 inches square or larger, to a stated percentage of the sample's initial height. Common IFD values are generated at 25 and 65 percent of initial height.

Support Factor (Compression Modulus)—Support Factor=65% IFD 25% IFD determined after one minute of rest or recovery. When the support factor is known it can be used in conjunction with a known 25% IFD value to determine the 65% IFD value. Seating foams with low support factor are more likely to bottom out under load.

Flex Fatigue (Dynamic Fatigue)—A softening or loss of firmness. In general, fatigue is measured in the laboratory by repeatedly compressing a foam sample and measuring the change in IFD. Several different protocols, including methods contained in ASTM, ISO, and BIFMA testing standards, may be used. Even ASTM 3574, one of the more widely used sets of testing standards, contains multiple testing methods. In selecting one or more test methods, it is important to consider the intended foam application and to choose a testing protocol that closely simulates the intended application.

Tear Strength—A measure of the force required to continue a tear in a foam after a split or break has been started and expressed in pounds per inch (lb./in.). This property is important in determining suitability of foam in applications where the material is sewed, stapled, or otherwise anchored to a solid substance.

Resilience—An indicator of the surface elasticity or “springiness” of foam. It is measured by dropping a steel ball onto the foam cushion and measuring how high the ball rebounds.

Hysteresis—Measurement of a foam's ability to maintain original support characteristics after flexing. Normally, the firmness (IFD) is tested at 25% indentation, 65% indentation and again at 25% on the way up. Hysteresis is the percent of 25% IFD loss measure as a compression tester returns to the normal (25% IFD) position after measuring 65% compression. Lower hysteresis values, or less IFD loss are desirable. Current research indicates that hysteresis values may provide a good indication of overall flexible foam durability. Low hysteresis in conventional foam is equal to less IFD loss in durability and fatigue tests.

Air Flow—Amount of air expressed in cubic feet per minute, that can be drawn through a 2″×2″×1″ foam sample at 0.5-inch water pressure differential. Air Flow is measured by a test. High air flow equates to very open foam while low air flow tends to be pneumatic. Air flow can be a critical factor in FPF performance depending on the application. For instance, in normal furniture cushioning applications, a pneumatic foam with low air flow will have poor durability and high compression sets, regardless of density. However, in a packaging foam, a foam with minimal airflow may be desirable as a shock absorber, or in medical applications, slow recovery foams are used to improve patient care. Low air flow foams are also used in gasketing applications in air conditioning and heating.

Details on the laboratory procedures used to measure FPF performance properties can be found in ASTM 3574, which is incorporated herein by reference, and in other testing standards such as those promulgated by ISO and BIFMA. Also, to make a fair comparison of FPF sample properties, the same test methods and testing conditions must have been applied.

Certain materials used in the construction of a mattress, such as, for example, the textile components (such as the ticking and fire barrier fabrics) and resilient cushioning materials (foams) may physically degrade over time. In the case of the ticking fabric, physical degradation may be compounded by aesthetic degradation as well as staining and surface abrasion may make the ticking appear worn before it has in fact suffered structural breakdown.

Different textile constructions and compositions respond differently to laundering factors such as washing temperature, drying temperature, detergent compositions and the like. These factors can affect the degree to which textiles shrink from their original size, change the feel or hand of the material or negatively impact the performance aspects of the fabrics as it relates to such dimensions as flame retardance or anti-microbial performance.

Mattress ticking is likely to become soiled during their service lives by a wide range of substances. Consumers will be inclined to attempt to launder the covers that are made from tickings if the design promotes their removal from the mattress core assembly, even despite possible warnings by manufacturers against laundering.

Even if laundering is not discouraged by the mattress manufacturer, the physical size of a ticking assembly that is not able to be physically separated may preclude the ability to fit it into many washing machines or dyers that consumers have in their homes.

It may be beneficial, both from a cost perspective (by delaying replacement cycle of entire mattress purchase) as well as an environmental impact perspective, to provide mattress designs that allow components to be reversed or replaced without replacing the mattress in its entirety, especially if laundering is not practical for any of the aforementioned reasons. The ability for a consumer to selectively reverse a mattress ticking assembly, may permit them to extend the anticipated usable life of the mattress in the event that one side of the mattress ticking became worn or soiled to an extent that cleaning, while possibly sanitizing the material, could not remedy aesthetic degradation. This is another clear benefit that the present disclosure has over the prior art.

Federal regulations clearly define what a mattress is and that it is subject to certain testing requirements, including 16 CFR 1633-Standard for the Flammability (Open Flame) of Mattress Sets. The ticking assembly depicted herein by the present disclosure is the outermost fabric assembly of the mattress as it is manufactured for sale by the mattress manufacturer and that is subjected to this testing described above.

In accordance with the definitions found in Section 2 of the Flammable Fabrics Act as amended (15 U.S.C. 1191) and 16 CFR 1633 Standard for the Flammability (Open Flame) of Mattress Sets, it has been discovered that a consumer-removable, detachable and reversible mattress ticking assembly can deliver a variety of benefits not heretofore provided in mattress design.

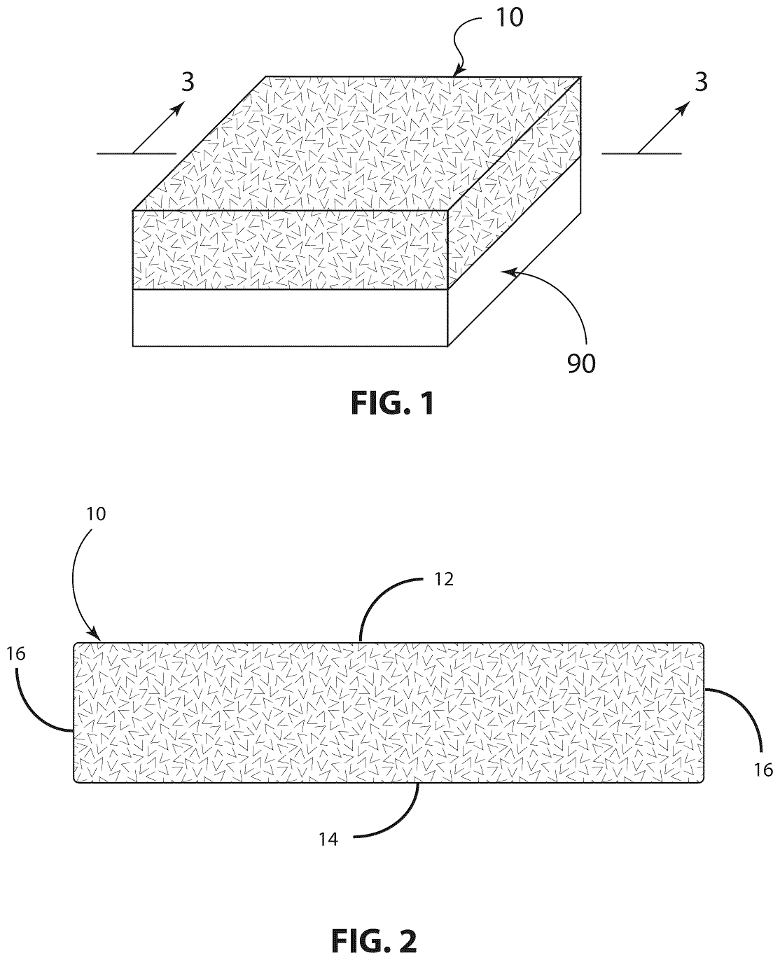

FIG. 1 illustrates a mattress assembly 10 supported on a mattress foundation 90 according to an embodiment of the present disclosure. Mattresses are often sold with a matched foundation. Foundations, also commonly referred to as the “box-spring” have a three-fold purpose: to raise the mattress's height, making it easier to get on and off the mattress; to absorb shock and reduce wear to the mattress; and. to create a flat and firm structure for the mattress to lie upon. Some mattress users elect to place their mattress on platform style bed frame to create the firm and flat surface for the mattress to lie upon. Other users simply place the mattress on the floor.

With reference to FIG. 2, mattress assembly 10 may include a top side 12 that is customarily referred to the top horizontal, panel face of the mattress assembly, a bottom side 14 that is customarily referred to as the bottom horizontal, panel face of the mattress assembly, and a peripherally extending side 16 disposed between top side 12 and bottom side 14 that are customarily referred to as the borders of the mattress assembly. The illustration of these directional references is provided to aid the clarity and understanding of the other descriptions that may appear herein.

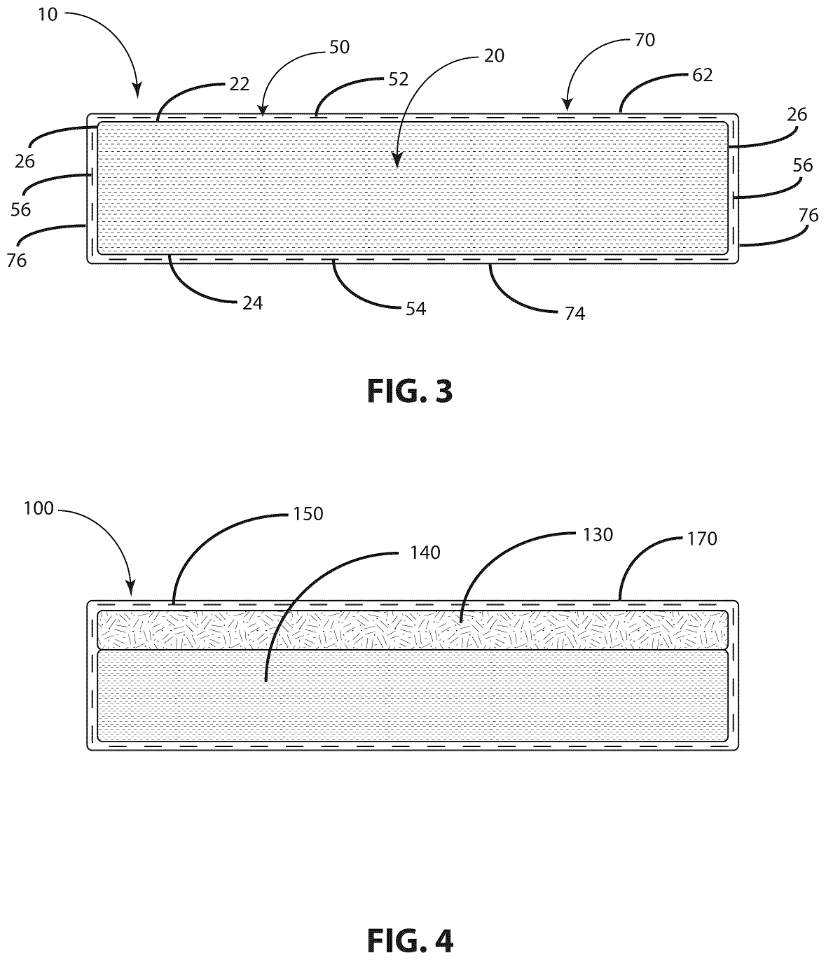

As shown in FIG. 3, mattress assembly 10 may include a core 20 having a single cushioning element, a fire barrier 50, and a detachable and reversible mattress ticking assembly 70, according to an embodiment of the present disclosure. As described in greater detail below, the various embodiments of the detachable and reversible mattress ticking assemblies of the present disclosure may further include a connector for releasably securing the ticking assembly in a first orientation with a reverse face facing the mattress core and an obverse face facing outwardly, and in a second orientation with the obverse face facing the mattress core and the reverse face facing outwardly.

For example, core 20 may include a top side 22 such as a top horizontal, panel face of the core, a bottom side 24 such as a bottom horizontal, panel face of the core, and a peripherally extending side 26 disposed between top side 22 and bottom side 24. The core 20 having a single cushioning element is optional and at least partially enclosed by fire barrier 50 that may be required for the composite mattress to comply with flammability regulations. The fire barrier 50 may have a top side 52, a bottom side 54, and a peripherally extending side 56 disposed between top side 52 and bottom side 54. The detachable and reversible mattress ticking assembly 70 encloses the core and fire barrier. The detachable and reversible mattress ticking assembly 70 may have a top side 72, a bottom side 74, and a peripherally extending side 76 disposed between top side 72 and bottom side 74. This embodiment, as well as others described herein, may apply to mattresses designed as either single-sided units (not “flippable” or intended to used only with the top side of the mattress assembly in contact with the user of the mattress and the bottom side of the mattress assembly in contact with the foundation or other supportive structure for the mattress) or two-sided mattresses (“flippable”). As explained below, mattress assembly 10 meets the flammability standards of 16 CFR 1633.

The specific detailed directional element descriptions provided for in the mattress assembly of FIGS. 1-3 are also present in subsequent drawings. In the interest avoiding redundancy only those particular elements that substantially differ from or are not included in the mattress assembly of FIGS. 1-3 will be explicitly identified in the following embodiments and corresponding drawings.

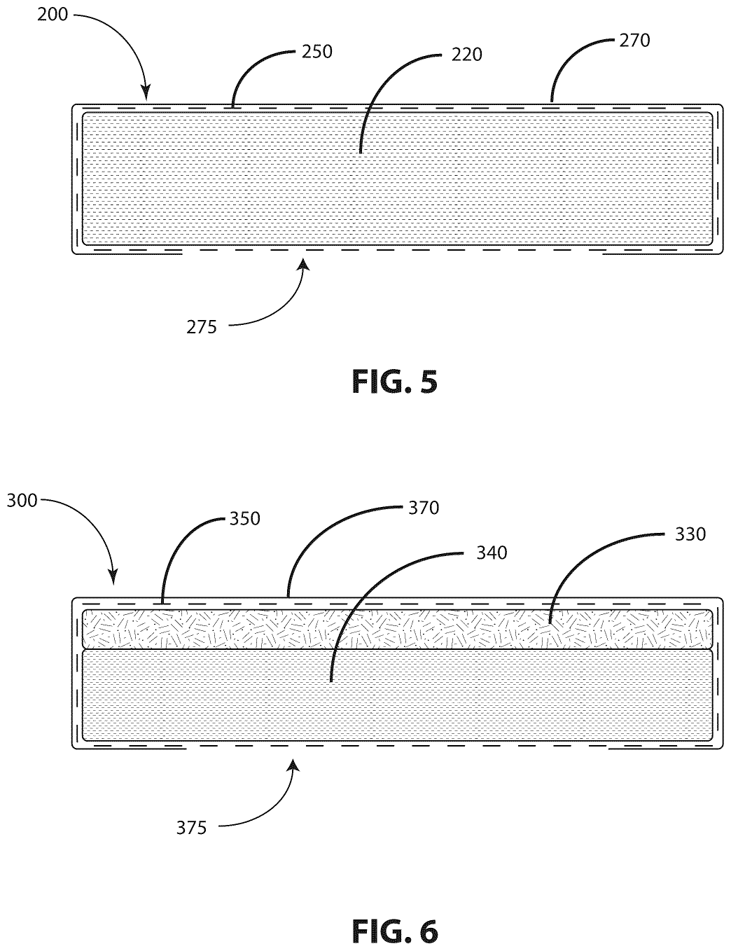

FIG. 4 illustrates a mattress assembly 100, according to an embodiment of the present disclosure. In this illustrated embodiment, mattress assembly 100 may include a core having two cushioning elements, for example, an upper disposed cushioning element 130 and lower disposed cushioning element 140. The core having two cushioning elements is optionally and at least partially enclosed by a fire barrier 150 that may be required for the composite mattress to comply with flammability regulations. A detachable and reversible mattress ticking assembly 170 in accordance with an embodiment of the present disclosure encloses the core and fire barrier.

It is fully contemplated by the present disclosure that the cushioning elements 130 and 140 may be selected from similar or dissimilar materials. Additionally, the present disclosure is not limited to any particular quantity or configuration of cushioning elements. The elements may be layered horizontally as illustrated herein or they may be an assembly of vertically oriented elements. An innerspring unit having of multiple attached or independent coils, whether pocketed or not, may be an example of a cushioning element with a vertical orientation. This design flexibility is demonstrated in this FIG. 4 but is also anticipated to be possible in all descriptions and illustrations contained herein and in the interest of clarity and avoidance of redundancy not necessarily explicitly disclosed herein.

As shown in FIG. 5, therein illustrated is a mattress assembly 200 having a core 220 having a single cushioning element. The core 220 having a single cushioning element is optionally and at least partially enclosed by a fire barrier 250 that may be required for the composite mattress to comply with flammability regulations. A detachable and reversible mattress ticking assembly 270 in accordance with an embodiment of the present disclosure only partially encloses the core and fire barrier. In this embodiment, the detachable and reversible mattress ticking assembly 270 is fashioned to completely cover a top horizontal, panel face of the mattress and four vertical, border faces of the mattress but an area 275 of the bottom horizontal, panel face of the mattress may remain uncovered by the detachable and reversible mattress ticking assembly 270. This embodiment may be more suitable for a single-sided mattress than a two-sided mattress.

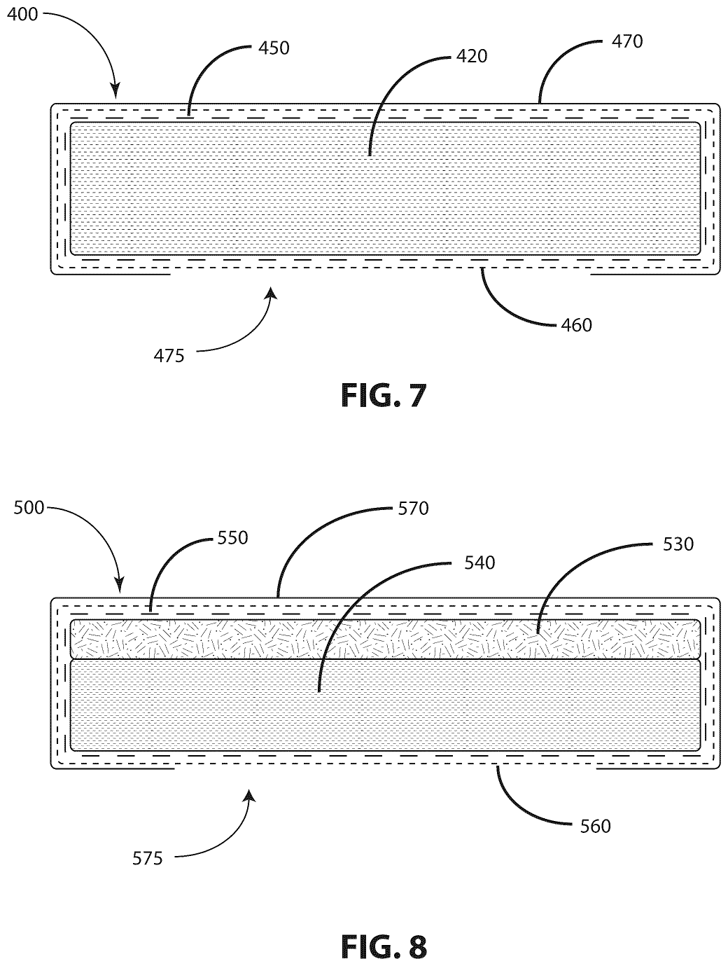

FIG. 6 illustrates a mattress assembly 300, according to an embodiment of the present disclosure. In this illustrated embodiment, mattress assembly 300 may include a core having two cushioning elements, consisting of an upper disposed cushioning element 330 and lower disposed cushioning element 340. The core having two cushioning elements is optionally and at least partially enclosed by a fire barrier 350 that may be required for the composite mattress to comply with flammability regulations. A detachable and reversible mattress ticking assembly 370 in accordance with an embodiment of the present disclosure only partially encloses the core and fire barrier. This embodiment may apply to both single-sided and two-sided mattresses. In this embodiment, the detachable and reversible mattress ticking assembly is fashioned to completely cover the top horizontal, panel face of the mattress and the four vertical, border faces of the mattress but an area 375 of the bottom horizontal, panel face of the mattress may remain uncovered detachable and reversible mattress ticking assembly 370. This embodiment may also be more suitable for a single-sided mattress than a two-sided mattress.

FIG. 7 illustrates a mattress assembly 400, according to an embodiment of the present disclosure. For example, mattress assembly 400 may include a core 420 having a single cushioning element. The core 420 having a single cushioning element is optionally and at least partially enclosed by a fire barrier 450 that may be required for the composite mattress to comply with flammability regulations. The core 420 and fire barrier 450 assembly is at least partially enclosed in a textile encasing 460. A detachable and reversible mattress ticking assembly 470 in accordance with an embodiment of the present disclosure only partially encloses the textile encased core and fire barrier. In this embodiment, the detachable and reversible mattress ticking assembly 470 is fashioned to completely cover the top horizontal, panel face of the mattress and the four vertical, border faces of the mattress but an area 475 of the bottom horizontal, panel face of the mattress may remain uncovered detachable and reversible mattress ticking assembly 470. The textile encasing 460 may be made from virtually any suitable material and is intended to either conceal the bottom side of the fire barrier 450 from view or access and to protect it from possible abrasion caused damage that may result from the open nature of the detachable and reversible mattress ticking assembly 470 that only partially encloses the core 420, fire barrier 450, and textile encasing 460. This embodiment may, similarly to FIG. 4, be more suitable for a single-sided mattress than a two-sided mattress.

With reference to FIG. 8, therein illustrated is a mattress assembly 500, according to an embodiment of the present disclosure. Mattress 500 may include a core having two cushioning elements, such as an upper disposed cushioning element 530 and lower disposed cushioning element 540. The core having two cushioning elements is optionally and at least partially enclosed by fire barrier 550 that may be required for the composite mattress to comply with flammability regulations. The core having two cushioning elements 530 and 540 that is covered by fire barrier 550 creates an assembly that is at least partially enclosed in a textile cover or textile encasing 560. A detachable and reversible mattress ticking assembly 570 in accordance with an embodiment of the present disclosure only partially encloses the textile encased core and fire barrier. In this embodiment, the detachable and reversible mattress ticking assembly 570 is fashioned to completely cover the top horizontal, panel face of the mattress and the four vertical, border faces of the mattress but an area 575 of the bottom horizontal, panel face of the mattress may remain uncovered. The textile encasing 560 may be made from virtually any suitable material and is intended to either conceal the bottom side of the fire barrier 550 from view or access and to protect it from possible abrasion caused damage that may result from the open nature of the detachable and reversible mattress ticking assembly 570 that only partially encloses the core, fire barrier and textile encasing 550. This embodiment may, similarly to FIG. 4, be more suitable for a single-sided mattress than a two-sided mattress.

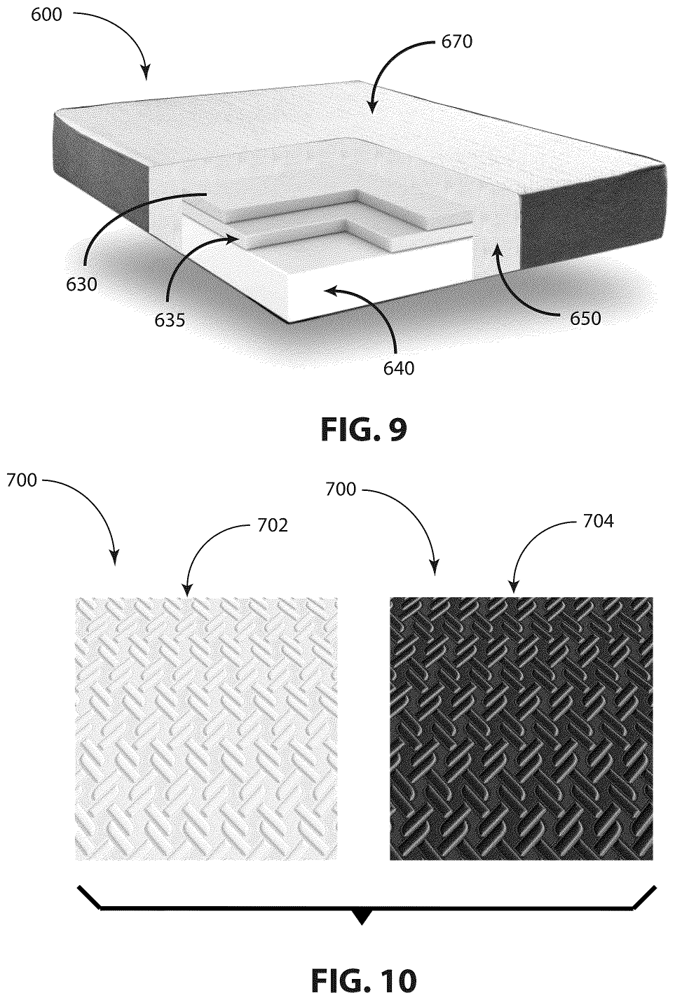

FIG. 9 illustrates a mattress assembly 600 according to an embodiment of the present disclosure. In this embodiment, mattress assembly 600 may include a core assembly having three cushioning elements, for example an upper cushioning element 630, a middle cushioning element 635, and a lower cushioning element 640 may be selected from similar or dissimilar materials. The cushioning elements may be detached from one another or joined together using techniques customary and accepted practices of the mattress or furnishing manufacturing industries. In this illustration, a fire barrier 650 is disposed around the outside of the assembly of the lower cushioning element 640, the middle cushioning element 635 and the upper cushioning element 630. The fire barrier encased core assembly is then completely encased in a detachable and reversible mattress ticking assembly 670 pursuant to the present disclosure. Optionally this design could also utilize a detachable and reversible mattress ticking assembly that only partially encloses the fire barrier encased core assembly.

With reference to FIG. 10, therein illustrated is a ticking fabric 700 according to an embodiment of the resent disclosure operable for use as a single ticking fabric or a plurality of ticking fabrics in forming the detachable and reversible mattress ticking assemblies described herein. For example, ticking fabric 700 may include an obverse face 702 and a different reverse face 704.

In this illustrated embodiment, the obverse face 702 of the ticking 770 and the reverse face 704 of the ticking 770 allows a detachable and reversible mattress ticking assembly to possess aesthetic and performance attributes that are unique to each side. The opposing planar faces 702 and 704 of the ticking 770 may possess similar performance attributes, dissimilar performance attributes or performance attributes opposite of one another, and/or the same or different appearance.

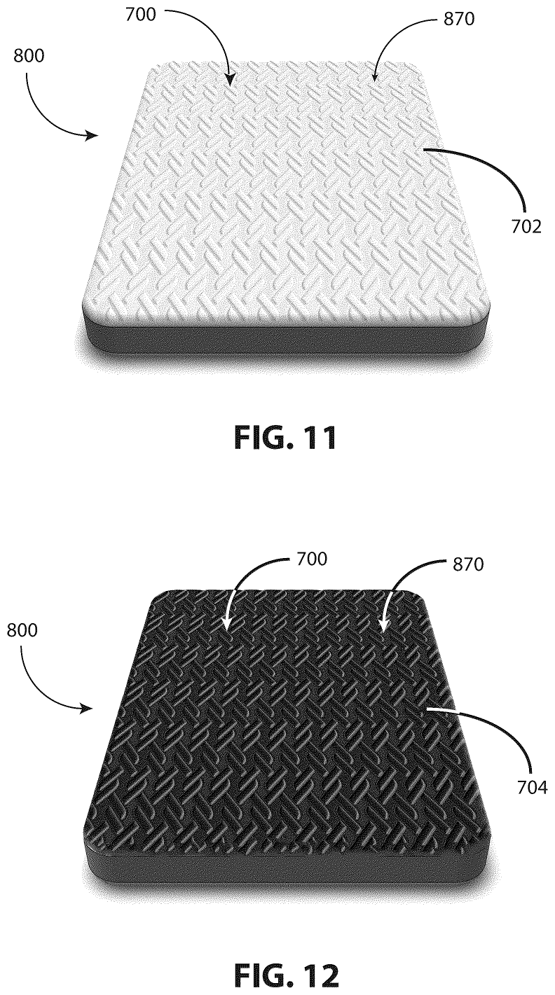

FIG. 11 illustrates a mattress assembly 800 with a detachable and reversible mattress ticking assembly 870 installed and formed from a single ticking fabric 770 onto a mattress core assembly (not shown in FIG. 11) with obverse side 702 disposed in a first or outermost orientation.

FIG. 12 illustrates mattress assembly 800 with detachable and reversible mattress ticking assembly 870 formed from the single ticking fabric 770 installed onto the mattress core assembly (not shown in FIG. 12) with reverse side 704 disposed in a second or outermost orientation.

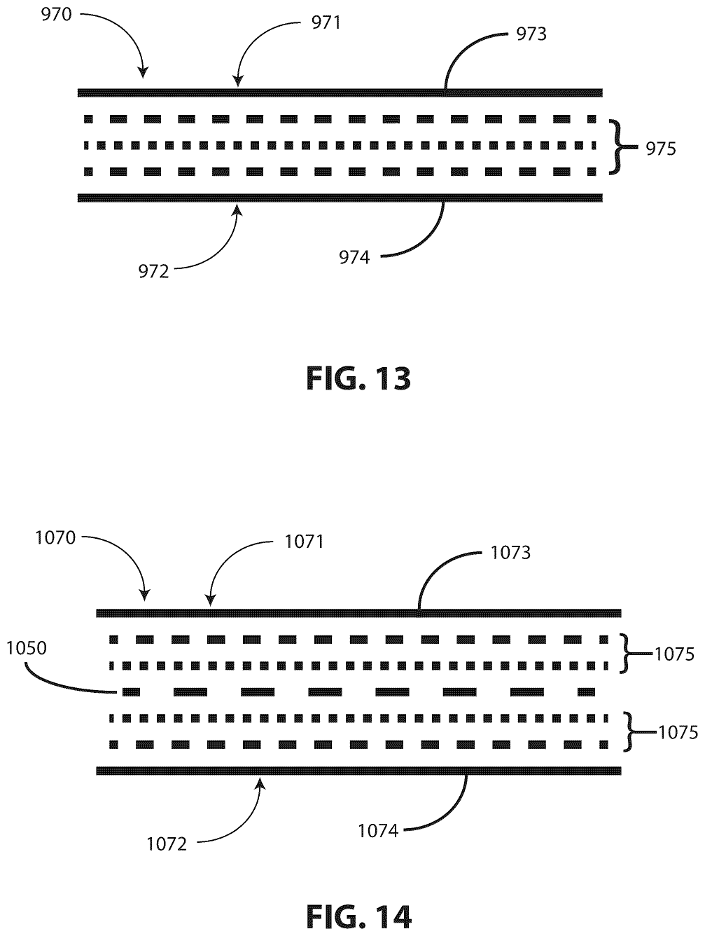

FIG. 13 illustrates a portion of a detachable and reversible mattress ticking assembly 970, according to an embodiment of the present disclosure, which detachable and reversible mattress ticking assembly 970 may be employed in the various mattress assemblies disclosed herein. In this illustrated embodiment, detachable and reversible mattress ticking assembly 970 may include a plurality of layers. For example, detachable and reversible mattress ticking assembly 970 may include a first outermost layer 971, an opposite second outermost layer 972, and one or more layers 975 disposed therebetween. First outermost layer 971 may define an obverse face 973, and opposite second outermost layer 972 may define a reverse face 974.

FIG. 14 illustrates a portion of a detachable and reversible mattress ticking assembly 1070, according to an embodiment of the present disclosure, which detachable and reversible mattress ticking assembly 1070 may be employed in the various mattress assemblies disclosed herein. In this illustrated embodiment, detachable and reversible mattress ticking assembly 1070 may include a plurality of layers. For example, detachable and reversible mattress ticking assembly 1070 may include a first outermost layer 1071, an opposite second outermost layer 1072, and one or more layers 1075 and a fire barrier 1050 disposed therebetween. First outermost layer 1071 may define an obverse face 1073, and opposite second outermost layer 1072 may define a reverse face 1074.

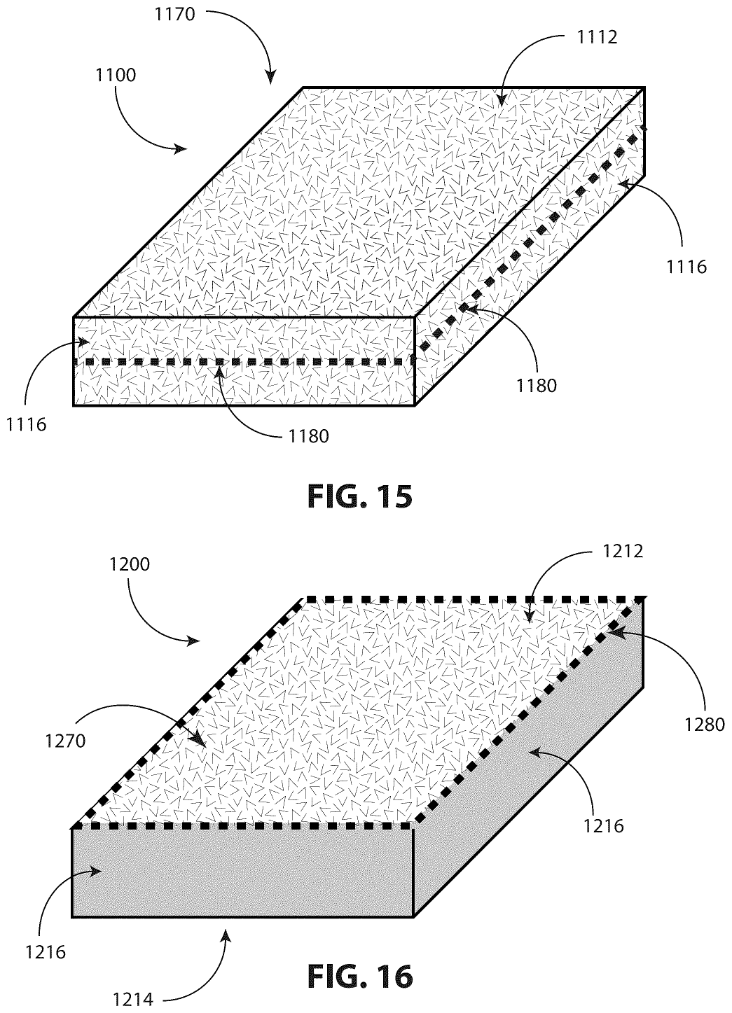

FIG. 15 illustrates a mattress assembly 1100 having a top side 1112 and a peripherally extending side 1116. Mattress assembly 1100 may include a detachable and reversible mattress ticking assembly 1170 installed onto a mattress core (not shown in FIG. 15). The present disclosure fully contemplates that the detachable and reversible mattress ticking assembly 1170 may be fashioned from one layer or fabric or a plurality of layers or fabrics, or a multiplicity of layers or fabrics. The assembly process for crafting the detachable and reversible mattress ticking assembly is free to locate seams or joining points of the selected fabrics at anywhere deemed to be aesthetically desirable and to meet the performance requirements of the finished composite article. For example, detachable and reversible mattress ticking assembly 1170 may incorporate a zipper 1180 or other closing mechanism suitable for joining elements of the detachable and reversible mattress ticking assembly. The location of the zipper 1180 may be in the peripherally extending side 1116 of the mattress assembly, which may facilitate the use on this embodiment on a two sided mattress.

FIG. 16 illustrates a mattress assembly 1200 having a detachable and reversible mattress ticking assembly 1270 with a reversible top side 1212 and a peripherally extending side 1216 operable to enclose a mattress core (not shown in FIG. 16). In this embodiment, only top side 1212 may be reversible and the peripherally extending side 1216 and a bottom side 1214 of the detachable ticking assembly need not be manufactured to be reversible. The reversible top side 1212 attaches to the peripherally extending side by a zipper 1280 or other closing mechanism typical for textile applications.

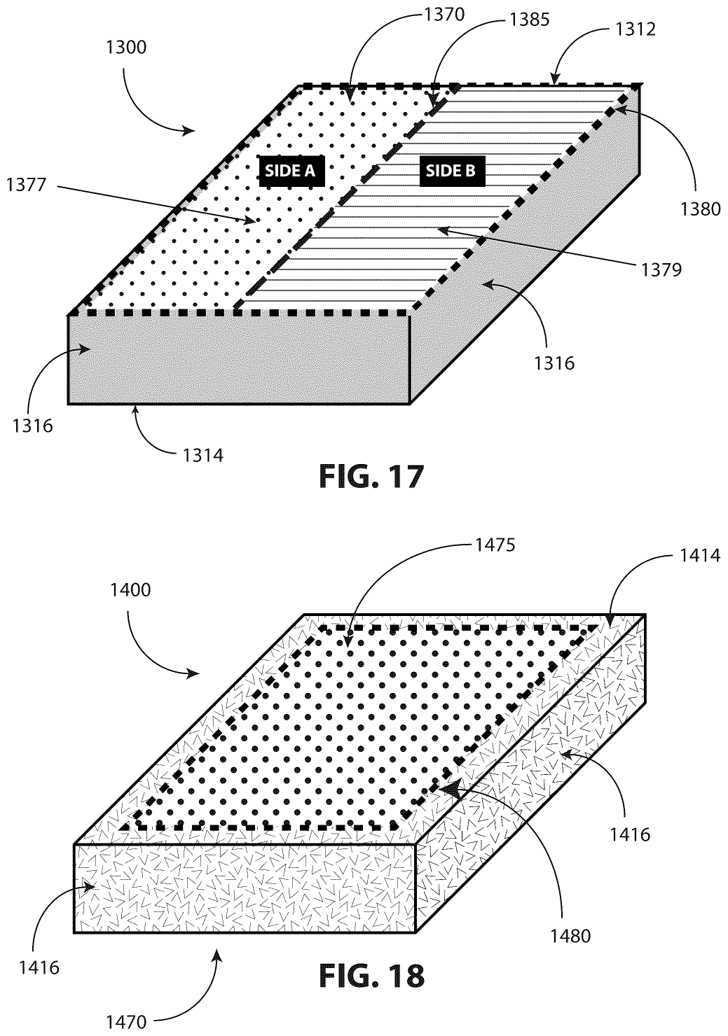

FIG. 17 illustrates a mattress assembly 1300 having a top side 1312 and a peripherally extending side 1316 operable to enclose a mattress core (not shown in FIG. 17). Mattress assembly 1300 may include a detachable and reversible mattress ticking assembly 1370 having a plurality of side-by-side portions 1377 and 1379. In this embodiment, only top side 1312 may be reversible and the peripherally extending side 1316 and a bottom side 1314 of the detachable ticking assembly need not be manufactured to be reversible. The reversible top side 1312 attaches to the peripherally extending side by a zipper 1380. A connector 1385 or other connecting mechanisms typical for textile applications such as stitching, snaps, hook-and-loop fasteners, adhesive, etc. may be used to attach adjacent portions of ticking portions 1377 and 1379. In this embodiment, top side 1610 may be reversible and the peripherally extending side 1316 and bottom side 1314 of the detachable ticking assembly are not manufactured to be reversible.

FIG. 18 illustrates a mattress assembly 1400 having a bottom side 1414 and a peripherally extending side 1416 operable to enclose a mattress core (not shown in FIG. 18). Mattress assembly 1400 may include a detachable and reversible mattress ticking assembly 1470. On the bottom side 1414 of the detachable and reversible mattress ticking assembly 1470 may include a base fabric 1475, which is typically less costly than the fabrics used on the top side (not shown in FIG. 18) and peripherally extending side 1416, to cover the bottom face of the mattress. The base fabric 1475 may cover a portion of the bottom of the mattress assembly, e.g., the peripheral edges of the base fabric 1475 may be spaced from the peripheral edge of the mattress assembly. A zipper 1480 or similar mechanism may be employed for the joining point of fabrics. The present disclosure fully contemplates that the detachable and reversible mattress ticking assembly 1470 may be fashioned from one fabric or a multiplicity of fabrics. The assembly process for crafting the detachable and reversible mattress ticking assembly is free to locate seams or joining points of the selected fabrics at anywhere deemed to be aesthetically desirable and to meet the performance requirements of the finished composite article.

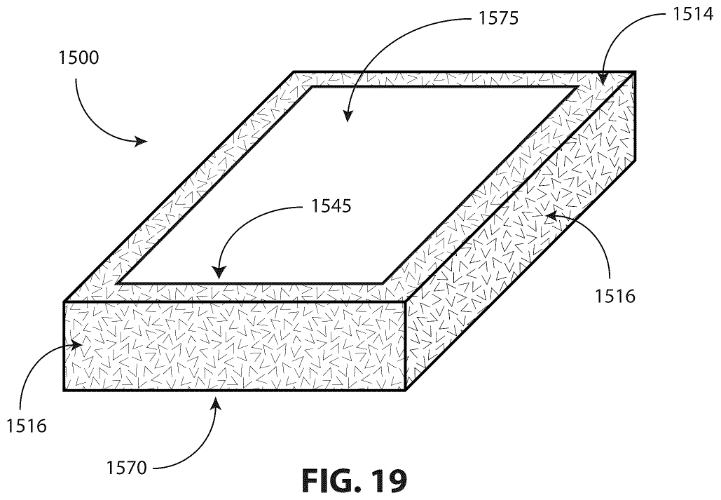

FIG. 19 illustrates a mattress assembly 1500 having a bottom side 1514 and a peripherally extending side 1516 operable to enclose a mattress core (not shown in FIG. 19). Mattress assembly 1500 may include a detachable and reversible mattress ticking assembly 1570 in a manner that partially encloses the mattress assembly 1500. On the bottom side 1514 of the detachable and reversible mattress ticking assembly 1570, the open edges of the fabric used to form the mattress ticking assembly may be finished with a hem 1545 or other suitable technique to lend a finished appearance to product and to mitigate potential that edges of the fabric used could fray or come apart. The hem 1545 may incorporate elastic material to promote it's the ability of the opening in the design to stretch open to facilitate installation over the mattress assembly and then to contract once installed to promote stability of the positioning of the ticking assembly 1570 while in use. This design embodiment that is open, e.g., an area 1575 of the bottom horizontal, panel face of the mattress may remain uncovered on the bottom side 1514. Such a configuration may be more suitable for a single-sided mattress. As will be appreciated, an elastic hem may be employed in other of the disclosed embodiments.

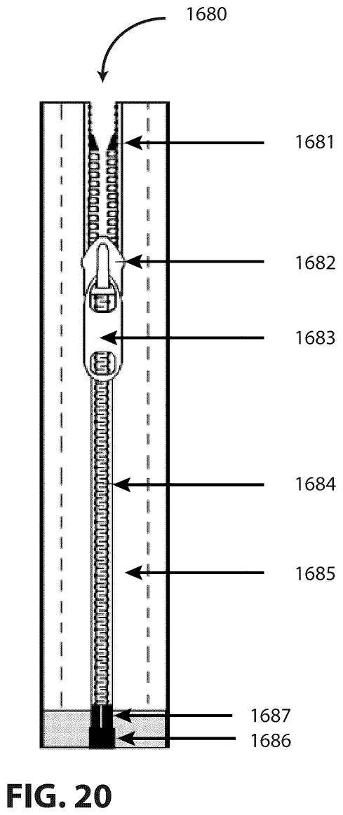

FIG. 20 illustrates a zipper assembly 1680 that may be suitable for use in the embodiments of the present disclosure. Zipper assembly 1680 may be a separating zipper, which includes a top stop 1681, a slider head 1682, a pull tab 1683, a chain 1684, zipper tape 1685, an insertion pin 1687, and a retainer box 1686. An attribute of the design of the detachable and reversible mattress ticking assembly disclosure recited herein is that when a zipper is utilized in the fabrication of the assembly, slider head 1682 and zipper pull 1683 may be specially selected so as to permit the opening and closing of the zipper when the detachable and reversible mattress ticking assembly is installed on the mattress in either the obverse or reverse orientation.

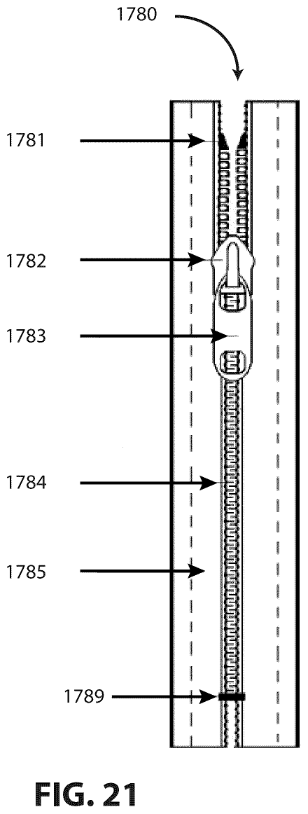

FIG. 21 illustrates a zipper assembly 1780 that may be suitable for use in the embodiments of the present disclosure. Zipper assembly 1780 may be a one end closed zipper, which includes a top stop 1781, a slider head 1782, a pull tab 1783, a chain 1784, zipper tape 1785, and a bottom stop 1789. An attribute of the design of the detachable and reversible mattress ticking assembly disclosure recited herein is that when a zipper is utilized in the fabrication of the assembly, slider head 1782 and zipper pull 1783 may be specially selected so as to permit the opening and closing of the zipper when the detachable and reversible mattress ticking assembly is installed on the mattress in either the obverse or reverse orientation.

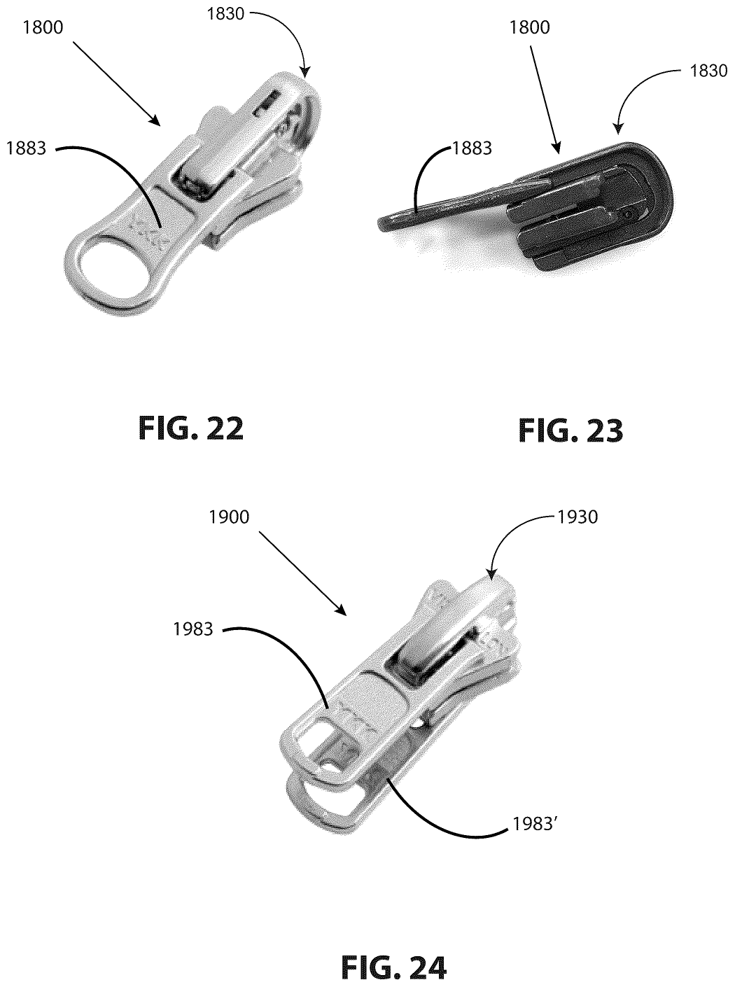

FIGS. 22 and 23 illustrate a zipper slider head 1800 suitable for use in the embodiments of the detachable and reversible mattress ticking assembly disclosed herein. Zipper slider head 1800 may include a body 1830 and a zipper pull tab 1883, which in this example can transit from an obverse face of the zipper slider head 1800 to a reverse face of the zipper slider head 1800.

FIG. 24 illustrates an alternative zipper slider head 1900 suitable for use in the embodiments of the detachable and reversible mattress ticking assembly disclosed herein. The zipper slider head 1900 may include a body 1930 having two sides, each side providing a pull tab to permit opening and closing of the zipper from either side, e.g., zipper slider head 1900 may include an obverse side pull tab 1983 and a reverse side pull tab 1983′.

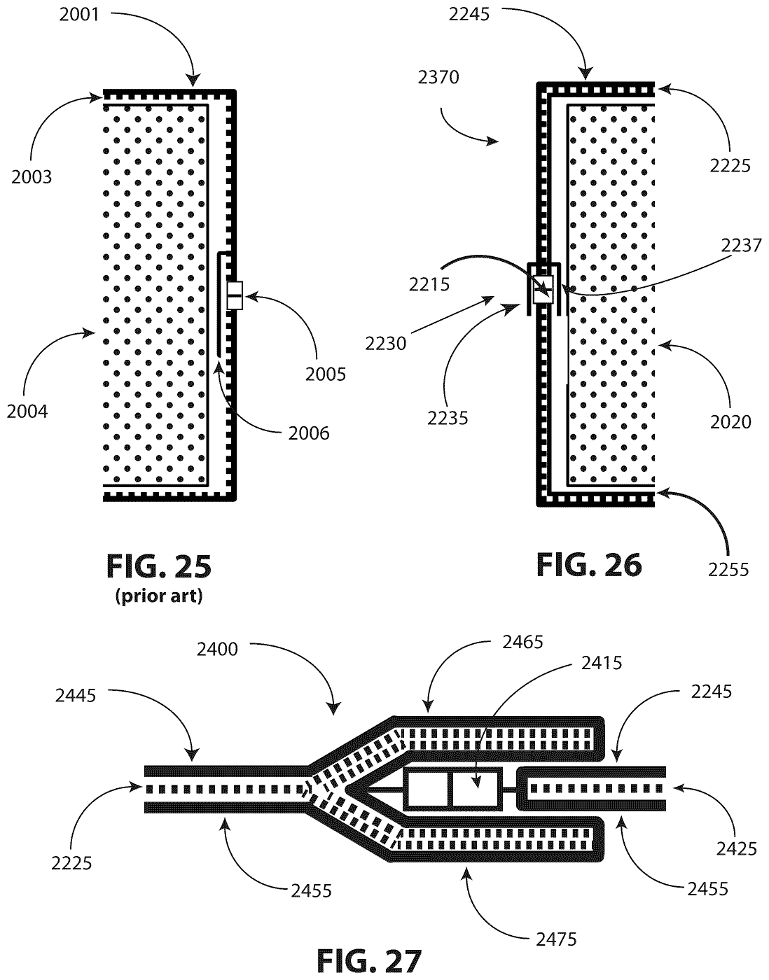

FIG. 25 illustrates a prior art mattress assembly. In the typical design approach, a mattress core assembly 2004 is encased in a ticking assembly 2001 made from a textile that incorporates a fire barrier 2003 that may be either unitarily and integrally formed with the textile or that is adhered to the ticking textile using customary textile techniques, such as gluing, laminating or sewing. A flame-retardant textile flange 2006 is disposed behind a void area created by the zipper 2005. The textile selected for the flange is chosen from textiles that are inexpensive and of a utilitarian appearance that may not be acceptable to be placed on the exterior, consumer visible face of the mattress.

In FIG. 26, a detachable reversible mattress ticking assembly 2370 according to an embodiment of the present disclosure is disposed around a mattress core 2020. For example, the detachable and reversible mattress ticking assembly 2370 of this embodiment may include of an obverse textile face fabric 2245, a fire barrier 2225, and a reverse textile face fabric 2255, and a connector 2230. The fire barrier 2225 may either be unitarily and integrally formed with one or both of the face fabrics 2245 and 2255, or it may have been manufactured independently of the face fabrics and disposed between them with any customary textile formation practices. Connector 2230 may be a zipper having a first textile flange 2235 and a second textile flange 2237, which flanges may provide a barrier in either orientation of the detachable and reversible mattress ticking assembly 2370 when installed on the mattress core, e.g., the first textile flange 2235 and the second textile flange 2237 reduces a point of vulnerability in the flame barrier coverage of the mattress fuel load by providing a barrier adjacent to the space or breach point where the fire barrier is non-contiguous and not present due to the zipper 2215.

FIG. 27 illustrates a connector 2400, according to an embodiment of the present disclosure. An obverse face fabric 2445 and a reverse face fabric 2255 are extended past the space or breach point created by a zipper 2415. The extension creates a flap 2465 over the obverse face of the zipper 2415 and a similar flap 2475 over reverse face of the zipper 2415. By extending the fire barrier 2425 within the flaps, the protective nature of the fire barrier 2425 now covers the space or void created by the zipper 2415. Each side of the zipper may include a flaps or pockets that extend over the zipper faces.

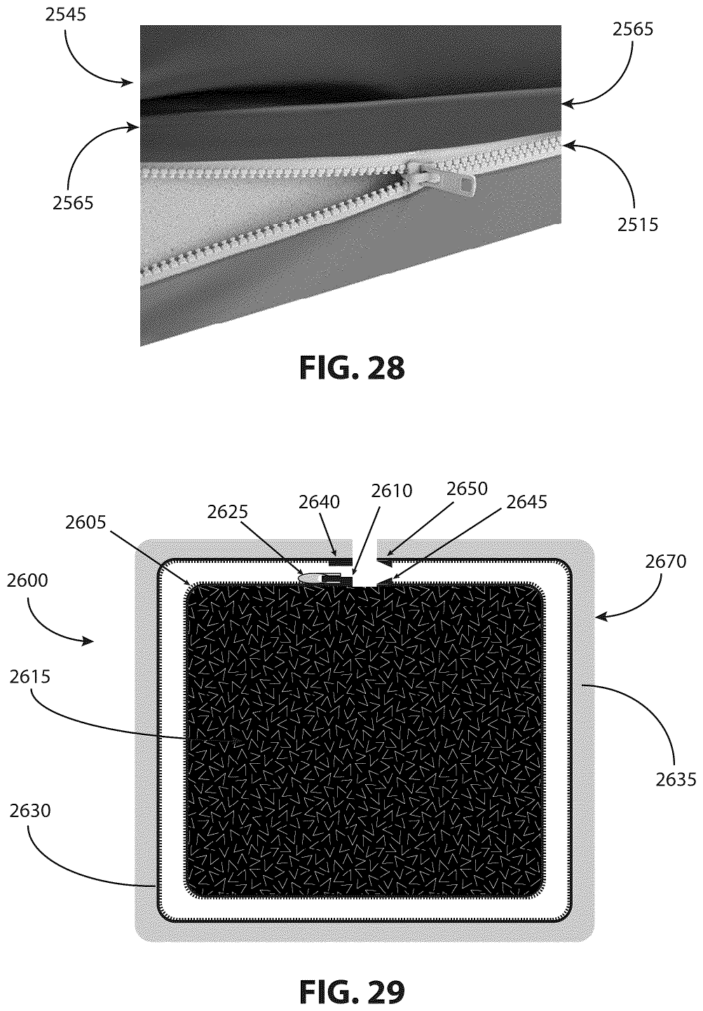

FIG. 28 illustrates an exterior view of a textile flap or waterfall zipper cover design, according to an embodiment of the present disclosure. A portion of obverse face fabric 2545 is fashioned into a flap 2565 that will cover the exposed area of the zipper 2515. For example, the flap 2565 is folded upward to reveal the zipper, but in normal use may fall down over the zipper 2515. In the design of the reversible ticking assembly contemplated herein, the reverse face fabric (not visible here) may be similarly configured with a second flap.

FIG. 29 illustrates a mattress assembly 2600 having a mattress ticking assembly 2670 and a mattress core (not shown in FIG. 29), according to an embodiment of the present disclosure. For example, the detachable and reversible mattress ticking assembly 2600 may be entirely reversible. In this illustrated embodiment, a first zipper tape (not shown in FIG. 29) with a first zipper chain 2605 and retainer box 2610 is attached to the outermost edge of the obverse top face 2615 of detachable and reversible mattress ticking assembly 2670 with a combined zipper slider head and pull-tab element 2625 installed onto the first zipper chain 2605. A corresponding second zipper tape (not shown in FIG. 29) with a second zipper chain 2630 is attached to the obverse face of the peripheral side 2635 of the detachable and reversible mattress ticking assembly 2670. The corresponding second zipper tape with the second zipper chain 2630 is positioned and oriented so as to match the position of the retainer box 2610 with an insertion pin 2640 in order to facilitate an intended function of the zipper. The alignment of first zipper tape with first zipper chain 2605 to second zipper tape with second zipper chain 2630 results in the proximate location of a first top stop 2645 of the first zipper tape with zipper chain 2605 to a second top stop 2650 of the second zipper tape with the second zipper chain 2630. This design approach may be suitable for detachable and reversible mattress ticking assemblies contemplated by the embodiments of the present disclosure where the entirety of the ticking assembly is designed to be detachable and reversible.

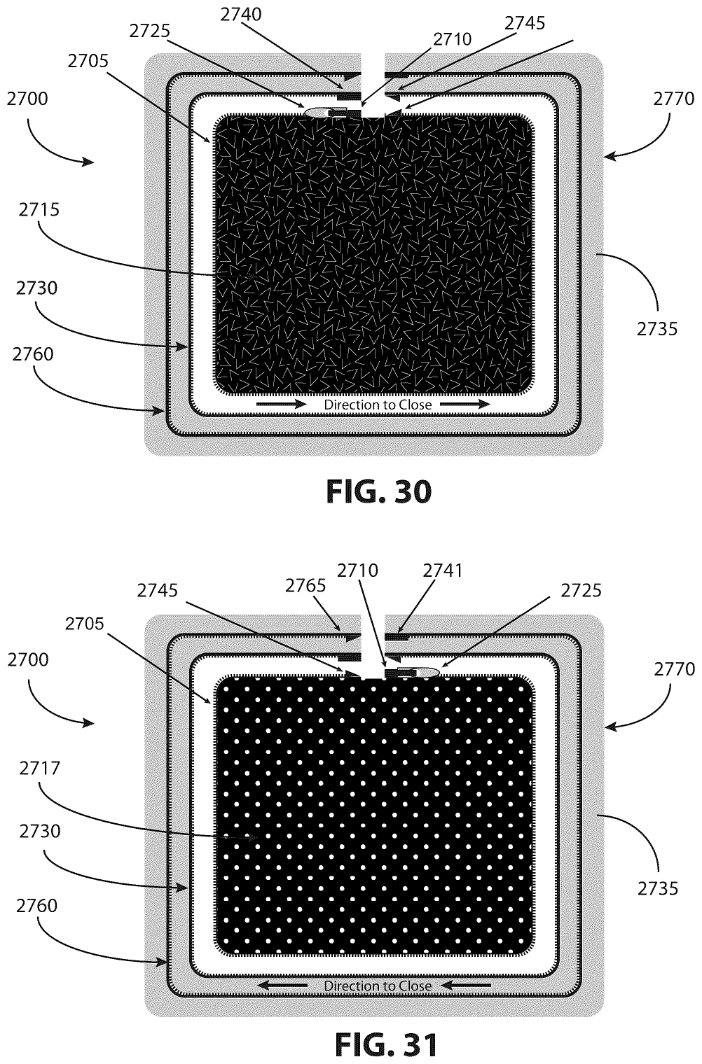

FIG. 30 illustrates a mattress assembly 2700 having a detachable and reversible mattress ticking assembly 2770 and a mattress core (not shown in FIG. 30), according to an embodiment of the present disclosure disposed in a first configuration. In this illustrated embodiment, the detachable and reversible mattress ticking assembly 2770 may be only partially reversible. For example, a zipper tape (not shown in FIG. 30) with a first zipper chain 2705 and retainer box 2710 is attached to the outermost edge of the obverse top face 2715 of the detachable and reversible mattress ticking assembly 2770 with a combined zipper slider head and pull-tab 2725 installed onto the zipper chain 2705. A second zipper tape (not shown in FIG. 30) with a second zipper chain 2730 and a third zipper tape (not shown in FIG. 30) and a third zipper chain 2760 are each attached to the peripheral side 2735 of the detachable and reversible mattress ticking assembly 2770. The spacing indicated in the drawing is provided for clarity and the second zipper tape with the second zipper chain 2730 and the third zipper tape with the third zipper chain 2760 may be in close proximity to one another. The corresponding second zipper tape with the second zipper chain 2730 may be positioned and oriented so as to match the position of the retainer box 2710 with an insertion pin 2740 so as to permit the intended proper function of the zipper in a first orientation. The alignment of the first zipper tape and first zipper chain 2705 to the second zipper tape with the second zipper chain 2730 results in the proximate location of a top stop 2745 of the first zipper tape with first zipper chain 2705 disposed adjacent to a top stop 2750 of the second zipper tape and second zipper chain 2730. The mattress ticking assembly 2700 in this drawing may be closed with a counter clockwise travel path (relative to the illustrated top view in FIG. 30) of the combined zipper slider head and pull tab 2725 that may interlock the teeth of the first zipper tape with first zipper chain 2705 with the teeth of second zipper tape with second zipper chain 2730. This action may permit to obverse top face 2715 of the mattress ticking assembly to be in an outward facing orientation when installed on the mattress core (not shown in FIG. 30). The third zipper tape and third zipper chain 2760 depicted herein remains unused when the obverse top face 2715 is attached in the aforementioned manner. This design approach can be suitable for detachable and reversible mattress ticking assembly contemplated by the present disclosure where the entirety of the ticking assembly is designed to be reversible.

FIG. 31 illustrates the mattress assembly 2700 having the detachable and reversible mattress ticking assembly 2770 with the top face 2715 (FIG. 30) disposed in an obverse orientation so that a top face 2717 faces outwardly. The corresponding third zipper tape with the third zipper chain 2760 is positioned and oriented so as to match the position of the retainer box 2710 with the insertion pin 2741 so as to permit the intended proper function of the zipper. The alignment of the first zipper tape and first zipper chain 2705 to the third zipper tape with the third zipper chain 2760 results in the proximate location of the top stop 2845 of the first zipper tape with the first zipper chain 2705 to a top stop 2765 of the third zipper tape and the third zipper chain 2760. The mattress assembly 2700 in this configuration may be closed with a clockwise travel path (relative to the illustrated top view in FIG. 31) of the combined zipper slider head and pull tab 2725 that would interlock the teeth of the first zipper tape with first zipper chain 2805 with the teeth of the third zipper tape with the third zipper chain 2760. This action permits the reverse top face 2717 of the mattress ticking assembly to be in an outwardly facing orientation when installed on the mattress core (not shown in FIG. 30). The second zipper tape and second zipper chain 2730 depicted herein remains unused when the reverse top face 2717 is attached in the aforementioned manner. This design approach can be suitable for detachable and reversible mattress ticking assembly contemplated by the present disclosure where the entirety of the ticking assembly is designed to be reversible.

Further to the above referenced illustrations and drawings, the following material variations are fully contemplated as selections that may be made to best commercially exploit the benefits of the present disclosure.

The single element mattress core of the disclosed mattress assemblies of the present disclosure may, in its most rudimentary form, be a homogenous block of polyurethane foam. The single element mattress core assembly may also be made of any variety of resilient and supportive cushioning materials customarily used in the construction of mattresses or upholstered furnishings. The single element mattress core assembly may be selected from foams, fiber or natural filling materials. Such examples of foams or resilient and supportive cushioning materials can include but are not intended to be limited to latex foam; reticulated or non-reticulated visco-elastic foam (sometimes referred to as memory foam or low-resilience foam), reticulated or non-reticulated non-visco-elastic foam; gel based foams; foams infused with graphite or other substances; high-resilience polyurethane foam; expanded polymer foams (e.g., expanded ethylene vinyl acetate, polypropylene, polystyrene, or polyethylene); and the like. Such foams and resilient cushioning materials are contemplated for use for all possible embodiments disclosed herein with regards to the present disclosure. The foam may preferably have a density of between 0.25 and about 10.0 pounds per cubic foot and a firmness that may range between 4 IFD and 80 IFD, as measured according to ASTM D3574. Fiber and natural filling materials may be selected from polyester fiber batting or wadding, cotton, wool, hair, coir, silk, cashmere, mohair or feathers.

The exemplary single element mattress core assemblies may be an assembled composite of a plurality of different cushioning elements that are glued, laminated or attached together to form a single unit. Utilizing different cushioning compositions or identical cushioning compositions with varied density or Indentation Force Deflection (IFD) can yield differential feel on each panel-oriented face of the core assembly element. One iteration of the internal foam formulations is a laminated foam structure having foam slabs layered and attached to each other in the following sequence: a 1″ natural latex with an IFD of 14, 1″ of latex with an IFD of 19 or higher, 2″ gel visco with an IFD range of 10-16 and 6″ of standard polyurethane foam with a density of 1.8 pounds per cubic foot and an IFD range of 25-40.

In the embodiments shown throughout, the single element mattress core assembly or multiple mattress core elements may incorporate a traditional mattress innerspring unit, whether made of metal or synthetic materials, such as plastic or polymers, with or without border rods, or pocketed coils or combinations thereof and the innerspring may be cushioned only on the top and bottom horizontal planar faces of the innerspring unit or alternatively encased in foam or other flexible, resilient cushioning materials. Other internal support mechanisms customarily utilized in the production of mattresses and articles of upholstered furniture are also anticipated as being able to be encased by the reversible ticking assemblies of the present disclosure. This use of a broad variety of materials to make the core assembly is fully anticipated for all potential embodiments to the present disclosure.

In the embodiments shown throughout, the single element mattress core assembly may also incorporate a traditional mattress innerspring unit, whether made of metal or synthetic materials, such as plastic or polymers, with or without border rods, or pocketed coils or combinations thereof and the innerspring may be cushioned only on the top and bottom horizontal planar faces of the innerspring unit or alternatively encased in foam or other flexible, resilient cushioning materials. Other internal support mechanisms customarily utilized in the production of mattresses and articles of upholstered furniture are also anticipated as being able to be encased by the reversible ticking assemblies of the present disclosure. This use of a broad variety of materials to make the core assembly and the virtually limitless combinations and permutations possible with this variety are fully anticipated for all potential embodiments to the present disclosure.

Similar material variations are contemplated for a mattress having a plurality of cushioning elements, such as the upwardly disposed cushioning element and lower disposed cushioning element, depicted in FIGS. 4, 6, and 8 or the lower cushioning element, middle cushioning element and upper cushioning element depicted in FIG. 9.

The scope of the present disclosure is not intended to be limited to the myriad of materials or fabrication approaches that may be used to form the fire barrier.

More specific examples for materials for us in fire barriers include aramids, including para-aramids (poly (p-phenylene terephthalamide), e.g., KEVLAR® (E.I. Dupont) and TWARON® (Teijin Twaron BV) and meta-aramids (poly (m-phenylene isophthalamide), such as Nomex® (E.I. Dupont); fiberglass; melamines such as BASOFIL® (BASF); poly-benzimidazole (PBI) (Celanese Acetate A.G); oxidized polyacrylonitrile (PAN); novoloids, such as KYNOL®. (American Kynol, Inc); pre-oxidized fibers and carbon fibers, modacrylics, such as, e.g., KANECERON® and PROTEX® (Kaneka), FR (fire- or flame-resisting, -resistant, -retarding or -retardant) rayon, FR viscose, such as, e.g., VERIFIBER® TCF FR Rayon (Ventex, Inc.), DFG® and FR Corona (Daiwabo Rayon) and LENZING FR® (Lenzing AG, Fibers Division), wool and FR-treated cotton. It should be noted that these fibers are merely exemplary, and other fire-retardant fibers that form a char, including fibers that are developed in the future may be used. Additionally, certain proprietary modacrylic fibers that release extinguishing/oxygen depriving elements such as antimony when exposed to an ignition source may be used. This chemical reaction may assist in snuffing out small flames that may occur on adjacent, non-FR components such as the mattress covering fabric or ticking. Blends that include at least one fire-retardant fiber that form a char may also be used. The blends may include one or more structure-providing char-forming fire-retardant fibers, FR-treated fibers, such as FR-treated polyester, and non-FR fibers. Accordingly, either the fibers themselves may have a chemical structure that provides flame retardant characteristics, or the fibers may include an additive that separately provides flame-retardant characteristics. Such additives may specifically serve to coat the fibers and provide a surface treatment and/or be dispersed within a given fiber network. Exemplary additives include polyphosphate compounds including polyphosphate emulsions, inorganic salts, aluminum oxides (e.g., Al2O3), boric acid compounds and/or halogen compounds.

The fire barrier of the mattress may be provided by woven, knitted textile or non-woven. Examples of possible woven structures may include plain weaves, taffeta weaves, oxford weaves, dobby weaves or other approaches known to skilled practitioners of textile weaving, and the types of knitted structures may include, but not be limited only to circular and warp knits, weft inserted knits, rib knits, jersey knits, interlock knits, double-knits, fleece knits, velour knits, and flat knits.

Non-woven fabrics used for the fire barrier may utilize a number of methods for maintaining the fibrous structures as well as joining more than a single fabric layer. Exemplary chemical bonding methods may include the use of adhesives, such as adhesive fibers, bi-component fibers or adhesive coatings that may be applied via powder coating, spray coating, baths, etc. Exemplary thermal bonding methods may include the use of low melting point fibers, bi-component fibers, point bonding, ultrasonic bonding, etc. Exemplary mechanical bonding may include carding, needle-punching, air-jet bonding, water-jet bonding, etc. In addition, the non-woven fabric for use may be three-dimensionally deformed via thermo-mechanical processes to form three-dimensional compressible features.

The textile encasing may similarly be formed by a myriad number of choices of textile formation approaches and compositional material selections without departing from the intent or scope of the present disclosure.

The textile encasing may optionally include flame retardant or resistant materials to augment or complement the aforementioned fire barrier, however the textile used may also be selected from a non-flame retardant or non-flame-resistant material. In addition to protecting the bottom side of the mattress fire barrier when the detachable and reversible mattress ticking assembly only partially encloses the mattress core assembly, the presence of the textile encasing may facilitate the installation and removal of the detachable and reversible mattress ticking assembly from the core assembly. Further the textile encasing may provide restriction of any lateral movement of the mattress core cushioning elements, particularly when a plurality of such elements is used in the mattress.

Suitable materials may be chosen from those well-known to mattress manufacturers as being sufficient to withstand the use demands of the intended application, available at costs that support their use in the end application and the aesthetic desires of consumers.

While the provisions and selections for the design and materials of the foregoing elements of the mattress design are typical of the types of materials conventionally used in the design and manufacture of mattresses and other articles of furnishing, it is the selection of materials and designs of the detachable and reversible mattress ticking assembly provided for by the present disclosure that represents the meaningful departure from customary mattress and furnishing fabrication approaches.

A requirement of the detachable and reversible mattress ticking assembly described herein be that the reversible element of the assembly, be it one part or the entirety of the assembly, possess a “retail-ready” appearance. That is to say that each face of the ticking assembly that is intended to be reversible should be tailored so that the end consumer is unable to distinguish whether the ticking assembly face that is outwardly visible was intended to be disposed to the interior or exterior of the mattress assembly. The “retail ready” appearance is accomplished by skilled finishing or tailoring of the textile assembly's sewn seams and hiding of unfinished edges of textiles used in the product.

Recent efforts in the engineered design of mattress ticking fabrics and by extension the marketing of mattresses have focused on integrating fibers, yarns and textile finishing agents into the ticking fabrics that are purported to deliver performance benefits to those sleeping on mattresses that contain such ticking fabrics. The present disclosure does not seek to validate or prove the efficacy of such product enhancements. It is sufficient for the present disclosure that such product enhancements have received commercial acceptance and have received demonstrated consumer demand.

The performance attributes sought to be delivered by these product developments include but are not limited to materials that draw heat away from the body and provide the sensation of cooling, materials that reflect heat back to the human body and provide the sensation of heating, materials that infused with aromas or scents that impart feelings of wellness or calm into the sleep environment and may even help control ailments, materials, such as copper and silver, that are purported that deliver therapeutic and pain relieving properties to the human body on the mattress and materials that possess anti-microbial performance properties that may reduce mold or fungal growth in the sleep environment. Many other such engineered textile enhancements

In addition to fiber, yarn and fabric treatments and finishes that may impart performance attributes into ticking fabrics that are desired by consumers, there are also textile formation techniques that may similarly create ticking fabrics that can be marketed as delivering desirable performance characteristics. For instance, textiles generally known as spacer fabrics may promote airflow and the perception of coolness. Another approach, such as textiles that are created to resemble fur may create the sensation or perception of warmth. Textile formation processes that create thickness out of yarns or fibers, such as textiles with nap including those like fleeces, velours, piles, velvets, and corduroys may create insulative structures that capture body heat and create a sensation or perception of warmth.

Exemplary cooling material include a metal, a metal alloy, a metal oxide, a carbon material, a ceramic material, a mineral, or combinations thereof. Reference in the industry often identifies these products as PCM's or phase changing materials.

Exemplary heating materials may include foil lined, or similarly reflective textiles. Heat-generating yarns and fibers combine tiny metal elements, which absorb body moisture and convert to heat energy, and insulating outer temperature to maintain warmth. Additionally, some warming fabrics may employ nano-materials and energized low-voltage DC (direct current) power sources.

Exemplary aroma infused materials may be selected from chemicals, compounds and essential oils that are regarded as possessing positive therapeutic properties in the realm of aromatherapy. Microencapsulation technologies and techniques may be employed to effect controlled release of such materials.

Exemplary materials that can provide therapeutic and pain-relieving properties may include copper and silver.

Exemplary anti-microbial agents are numerous and widely known in the textile industry. Such agents may be in the form of chemical treatments that are applied to fibers or yarns, inherent treated fibers and yarns where the desired chemistries are introduced to the fibers and yarns during their manufacturing formulation processes. Even certain metals, such as silver are known to possess desirable anti-microbial properties.

The proliferation of so many desirable performance attributes into the textiles that may be used as mattress ticking presents consumers with potential choice conflict that the present disclosure seeks to alleviate and overcome. The fashioning of a detachable and reversible mattress ticking assembly for use in the original manufacture of mattresses provides the previously unavailable opportunity to have one desirable performance attribute, such a cooling, reside in the same mattress as an opposing performance attribute, such as warming.

By fashioning a detachable and reversible mattress ticking assembly with a textile possessing cooling properties on the obverse face of the assembly and a different textile possessing warming properties on the reverse face of the assembly, the opportunity, heretofore unavailable to consumers, has been created that will enable them to buy a mattress with two distinct and opposing performance capabilities built into the same original product.

In the case of a cooling/heating construction, the consumer could elect to seasonally temperature regulate their sleep environment. In the warmer months of summer, they could use the ticking assembly with the cooling side disposed in the outermost position. In the cooler winter months, they could remove the ticking assembly, reverse the orientation and dispose the warming side to the outermost position.

An additional capability contemplated for the present disclosure may even be one that used differing colors of textile for the obverse and reverse faces of the detachable and reversible mattress ticking assembly. While in use, mattresses are nearly always covered with bed linens and their ticking is not readily visible. However, there is substantial investment made by mattress manufacturers in the visual appearance of mattresses and this visual appearance is a key to the selling proposition to consumers. Offering consumers the ability to “change the color of their mattress” is something not previously offered, but a selling attribute that could create positive acceptance in the marketplace.

Since the fabrication of the reversible ticking assembly may employ tailoring techniques found in garment fabrication, the outward aesthetic appearance of the mattress may not be diminished by the selection of either presentation of the ticking assembly.

While the selection of opposing or dissimilar performance or aesthetic attributes and delivery to consumers of such configurations is an improvement over the prior art, an additional improvement can also be achieved even if the obverse and reverse faces of the present disclosure are selected with performance attributes that are similar or even identical.

As previously observed, mattress ticking may become soiled or worn during use. The ability to launder a ticking assembly may not be feasible. In the past such staining or wear on a mattress ticking that was permanently installed on the product or on a removable, but one-sided and non-reversible mattress ticking might precipitate the replacement of entire unit prior to the end of its actual useful life. The present disclosure provides, in contemplated embodiments where the aesthetic and performance attributes of the obverse and reverse ticking faces are identical, consumers with the opportunity to simply reverse the detachable and reversible mattress ticking assembly and effectively prolong the useful life of the article.

The design approach of the exemplary embodiments recited herein may use relatively simple, single layer fabrics or fabric textile combinations and structures, however it is anticipated that more complicated, composite textile structures, such as a quilted, multi-layer assembly of decorative fabrics and thin foam sheets or fiber-based “quilting” fabric may be used in lieu of simple, single layer fabrics. Such textile structures may also include non-woven backer fabrics that assist in preparing such quilted, multi-layer assemblies and such “quilt assemblies” are well-known to the mattress manufacturing community and their use may not constitute a material departure from the present disclosure.

One desirable and efficient manner for crafting the primary ticking fabric of the present disclosure is to use double knitting techniques that integrally and unitarily form a knitted fabric with two distinct faces. The fabric is knitted so as to dispose cooling yarns and fibers on one face or side of the fabric and warming yarns on the other face or opposite side of the fabric. While there may some penetration of yarns from one side to other if employing this fabrication approach, the knitting operation is organized to have the majority of yarns or fibers on one face to constitute the minority of yarns on the opposite face and vice-versa. A three-layer knit is also contemplated with a middle layer that includes filler yarns or materials that serve to isolate one face from the other. The filler yarns or materials may include flame retardant materials that may be sufficient in and of themselves to enable the mattress to meet appropriate flammability standards.

The term “relief” is used in art to refer to a physical projection from a supporting background, usually a plane surface. The use of certain textile production processes, such as double knitting techniques, can permit a unitarily and integrally formed textile to have a pattern or texture on one face side of the fabric that projects outward from the planar surface of the fabric. This is the equivalent of a “relief”. It can be considered a “positive relief” for purposes of the present disclosure. On the back or opposite side of the fabric there may be a corresponding image or pattern that is displayed by the yarn configurations needed to create the relief but on the opposite side it may take on more of a “negative” aspect-similar to a photographic negative image in some regards. This constraint on the design approach can be overcome by judicious design choices.

The outward projection may be termed a “positive relief” and an inward projection or carving into the background or plane surface could Reliefs are classified according to the height of the figures' projection or detachment from the background. In a low relief, or bas-relief (basso-relievo), the design projects only slightly from the ground and there is little or no undercutting of outlines. In a high relief, or alto-relievo, the forms project at least half or more of their natural circumference from the background and may in parts be completely disengaged from the ground, thus approximating sculpture.

The use of double knitting techniques can also permit a unitarily and integrally formed textile to serve as a single fabric for use as the ticking in the present disclosure and the fabric may have a pattern or texture on one face side of the fabric and a pattern or texture on the other face side of the fabric.

Traditionally, ticking fabrics for mattresses have been yarn-based textiles that have been woven or knitted and form the outer surface of the mattresses. Woven fabrics have historically been used on innerspring mattresses, however the use of knitted fabrics as mattress ticking has increased in concert with the increase of non-innerspring mattresses and the use of expensive, high quality cushioning materials whose comfort benefits are diminished or hidden by woven fabrics and promoted and enhanced by knits. Woven fabrics are limited in their ability to stretch and recover, whereas knitted fabrics provide stretch and recovery properties after compression to provide support that conforms to the use of resilient cushioning materials in the mattress core assembly. The complete range of woven structures is anticipated, including, but not limited to simple and compound weaves, plain weaves, oxfords, taffetas, dobby weaves, drill, twill, gabardine and the like and these fabrics may also be greige, finished, uncoated or coated.

The scope of the present disclosure is not intended to be limited in any fashion by the wide range of textile formation techniques available to those seeking to exploit the benefits of this approach as described. A single, two-sided fabric that is integrally and unitarily formed by textile production methods may be suitable to convey the full range of benefits contemplated herein. Similarly, two or more independently formed textiles may be joined together by customary means, including sewing, quilting, thermally bonding, laminating and the like to serve as the ticking in the assembly described herein.

The ease of use by consumers of the detachable and reversible mattress ticking assembly of the present disclosure that uses a zipper as the closing mechanism is anticipated to be promoted using zipper mechanisms that are specifically engineered to be used in reversible textile structures. It is within the scope of the anticipated embodiments that such a specific zipper might not be used and instead a standard, single sided zipper might be employed. Such a selection may not cause the item to depart from the intent of the current disclosure. Additionally, other typically used textile closing mechanisms, such as buttons, snaps, hook and loop structures, and the like may be employed without deviation from the present disclosure.

Exemplary fibers used in the textiles formed for use as ticking in mattresses and mattress foundations may include, but are not necessarily limited to organic, natural, man-made or synthetic materials, including but not necessarily limited to cotton, nylon, rayon, wool, linen, silk, acrylic, olefin, polypropylene, polyesters, bi-component polyesters and elastomeric materials. The utility of the present disclosure may also benefit from the future introduction of future, novel textile production materials and incorporation of such materials is not anticipated to fundamentally alter or even improve the benefits to consumers that this disclosure conveys.

Further, the, detachable and reversible mattress ticking assembly of the present disclosure may include an elastic fibrous assembly. As used herein the elastic fibrous assembly is the combination of constituent fibers or fibrous components that have been fitted together to form a structure that exhibit recovery when a mechanical stress is applied to the structure and subsequently released. In particular an elastic nonwoven structure or weft-type circular knitted textile structure may be an elastic fibrous assembly. The elastic fibrous assembly may utilize a number of methods to intermingle elastic materials with fibrous assembly fire blocking materials. The elastic materials may be impregnated into, disposed or sprayed on, laminated or intermingled with fire blocking materials.

Elastic or elastomeric materials used herein may include for example, elastomeric polymers, such as polyurethane, chloroprene, etc. Polyurethane may include spandex fiber which may be understood to include segmented polyurethane, Spandex®, Dorlastan®, Lycra®, polychloroprene (a type of synthetic rubber also known as neoprene and other elastomeric type polymers that may include polyisoprene, polybutadiene, polystyrene-butadiene and silicones.

### Specific Embodiments

A twin sized embodiment of the present disclosure was prepared in the following manner:

A single foam core assembly may include two pieces of foam glued together was selected. One piece of standard polyurethane foam with a density of 1.8 pounds per cubic foot, an IFD of 38, and measuring 38″×74.5″×7″ was selected to serve as the bottom of the foam core assembly. A second piece of gel-infused, visco-elastic memory foam with a density of 3.0 pounds per cubic foot, an IFD of 10 and measuring 38″×74.5″×3″ was selected to serve as the top of the foam core assembly. The foam pieces were aligned and glued together in an industry accepted manner.

The glued together, also referred to as “laminated”, foam core assembly was covered in a tubular, circular knitted fire barrier fabric having corespun yarn formed from continuous filament fiberglass yarn, totally chlorine free FR rayon staple fibers and nylon staple fibers.

The fire barrier encased laminated foam core assembly was then encased or “loaded into” in a detachable and reversible mattress ticking assembly that fully enclosed the core assembly.

The detachable and reversible mattress ticking assembly was made in the following manner:
a. A textile with cooling property was selected to serve as the obverse face of the ticking.
b. A textile with warming property was selected to serve as the reverse face of the ticking.
c. The obverse and reverse face ticking materials were cut into customary pattern shapes of similar sizes for creation of a mattress ticking assembly of the intended size.
d. The ticking assembly design selected for this evaluation disposed these materials with opposing performance attributes on the entirety of the top side of mattress, the entirety of the peripherally extending sides of the mattress and approximately 2 inches in from each edge into the bottom side of the mattress.
e. A standard non-skid decking material of a stitch-bonded material was selected for the bottom side of the mattress ticking assembly to fill the area that may be left uncovered by the combined heating and cooling fabric ticking assembly portion.
f. The assembly of the textile pieces was performed to fashion a mattress ticking assembly that had a zipper joining the ticking fabric with the non-skid, stitch-bond material in a way that.

The finished mattress assembly was repeated seven times to make a total of eight identical specimens. Five of the specimens were made with the obverse face disposed in the outermost orientation and three of the specimens were made with the reverse face in the outermost orientation.

The alternate presentation of the mattress ticking with its reversible capability is part and parcel of the product's original sale into commerce. Mattresses must comply with the provisions of 16 CFR 1632.4 and 16 CFR 1633 to be sold legally in the United States. The alternate presentation capability of the present disclosure creates the likely possibility that the mattress may perform differently when subjected to full scale flammability testing depending on which face of the reversible ticking assembly is oriented in the outermost position. Therefore, unlike all other mattresses sold currently, this mattress design using the detachable and reversible mattress ticking assembly of the present disclosure may require complete validation and testing for each possible orientation.

Four specimens, two of each orientation, were subjected to testing for resistance from soldering ignition source under the protocol of 16 CFR 1632.4 (colloquially referred to as the ‘cigarette ignition standard”). The performance observed was objectively comparable and supported use of ticking substitution consideration for the design under the full-scale open flame testing to subsequently be conducted.

Since 16 CFR 1632.4 requires testing of two mattress panel surfaces for each mattress, the testing of four specimens, two of each orientation, may be expected to satisfy the intent and spirit of this regulatory requirement. If the results of this testing had been different and demonstrated a different classification for the ticking under testing, then the rules related to ticking substitution under the law may have necessitated additional 16 CFR 1633 testing.

Three specimens with the obverse face in the outermost orientation were subjected to testing for resistance to open flame ignition source under the provisions of 16 CFR 1633. Successful conformity to the passing criteria establishes a “qualified prototype”.

As the 16 CFR 1632.4 testing demonstrated that the ticking assembly performance was objectively comparable, then only one specimen with the reverse face in the outermost orientation had to be subjected to testing for resistance to open flame ignition source under the provisions of 16 CFR 1633 and this is done as a “subordinate prototype”. If, however, the 16 CFR 1632.4 results for each orientation had been different and demonstrated different classification for the ticking, then each orientation may have needed to be tested with three specimens to establish two “qualified prototypes’—one for each orientation.

These testing requirements for mattresses and the potential complexity of the decision processes related to possible testing outcomes is a key element of appreciating how this disclosure departs significantly from the prior art of reversible textile based articles that may be used in garment fabrication as the flammability performance of each side must be evaluated.

Throughout this description, in the interest of descriptive simplicity, only the term mattress has been used to describe the disclosure in terms of articles intended for sleeping upon. It is fully contemplated and anticipated the present disclosure is applicable to and offers benefit for the construction of mattresses and the construction of mattress foundations as defined herein. Any reference in the above specifications to the term mattress is equally intended to be applicable to the design of mattress foundations as well. With limited modification to the descriptive terms used in reference specifically to mattresses and mattress foundations, the present disclosure can also apply to other furnishing applications, including upholstered furnishing and transportation seating. For example, the detachable and reversible ticking assembly may be suitably employed on a mattress foundation.

The present disclosure relates to a ticking assembly, which includes the outermost layer of fabric, fabrics and/or related material that encloses a core and upholstery materials of a mattress or mattress pad. A mattress ticking or assembly of a mattress ticking and certain cushioning elements may include several layers of fabric or related materials quilted together or it may merely be a single textile item fashioned so as to encase fully or partially the core elements of the mattress.

It is important to note that the reversible ticking assembly depicted in the present disclosure is an integral part of the mattress and is not something that is added on to a mattress by a consumer at a date subsequent to original purchase.

It fully anticipated by the present disclosure that the textile example recited herein should not be limited to the particular fabrics described and that any materials or fabrics customarily or reasonably employed in the manufacture of mattresses or articles of upholstered furniture may be utilized to derive the benefits of the present disclosure.

The individual component materials used to construct a mattress may be drawn from the wide range of compositional and design approaches presently employed in the fields of mattress and upholstered furniture manufacturing without departing from the benefits being provided by the present disclosure. For instance, the use of antimicrobial treatments or finishes, such as those intended to mitigate or prevent bacterial or fungal growth, to provide odor-control or other similar benefits, on mattresses and upholstered furniture articles, including the fibers, yarns, filling materials, and foams, used to construct the mattresses or upholstered furniture articles may be anticipated by the present disclosure Other treatments or finishes, such as those intended to mitigate or prevent staining, soiling, moisture vapor penetration, moisture penetration, fluid penetration, or static electricity buildup are similarly anticipated.

The mattress that is intended to be covered by the ticking assembly disclosure disclosed herein is not intended to be limited and may be of any type, dimension, and/or shape. For example, the mattress may be a foam mattress, a coiled mattress, a foam and coil mattress, an air mattress, combinations thereof, or the like. Typically, the mattress is square or rectangular-shaped and has a thickness ranging from about 4 inches to about 20 inches. The length and width can vary depending on the intended application and typically has a width of about 2 feet to about 7 feet and a length of about 4 feet to about 10 feet, although custom sizes may require smaller or larger dimensions.

The mattress while typically being square or rectangular, may also take any other shape, including but not limited to round, triangular, oblong, trapezoidal, or elliptical.

Regulation mandating full-scale resistance to open flame and smoldering ignition sources has necessitated the addition of fire barrier such as fabrics as textile constructions located between the filling materials. These textile constructions may be independent of the ticking or may be integrated into the ticking by means of unitary textile formation or by adhering the fire barrier to the ticking. An optional means to providing for the fire-resistant properties of the fire barrier fabrics may be to impart flame retardant or resistant chemical treatments or finishes to the ticking, the filling material or both.

There are potential limitations and design considerations that are created by electing to integrate the fire barrier into the ticking assembly, either as a unitarily formed textile construction or as a part of an assembly of textiles. One design consideration is that sewing threads for joining fabrics used to make the ticking assembly should be selected from threads or yarns that are resistant to flame or other potential ignition sources. Para-aramid sewing threads may be one example of such selections. Another design consideration may be the need to dispose fabrics or materials that are flame or ignition source resistant behind any gaps in the fire barrier containing ticking assembly that may be created by the placement of zippers or the like that permit access or the ability to open the ticking assembly. One customary practice in current design is to place a flange of flame retardant non-woven materials behind the opening caused by zipper placement. For the detachable and reversible ticking assembly of the present disclosure, the flange-style design may have to be suitably engineered to promote both the aesthetic of the reversible design and the performance oriented necessity of isolating the fuel load of cushioning materials from exteriorly disposed ignition sources.

A1. A mattress assembly having a mattress core having a top side, a bottom side, and a peripherally extending side or sides disposed between said top side and said bottom side, and a detachable and reversible mattress ticking assembly for at least partially covering said mattress core. The detachable and reversible mattress ticking assembly having one or more textiles that are joined together to create an obverse face and a reverse face of the assembly, and the mattress assembly meets the flammability standards of both 16 CFR 1632.4 and 16 CFR 1633 when tested with the detachable and reversible mattress ticking assembly disposed in each of the possible orientations, specifically with both the obverse face of the assembly and the reverse face of the assembly each being disposed outwardly toward the end user of the mattress.

A2. The mattress assembly of claim A1, wherein the obverse face and the reverse face of the assembly are selected from a single textile or plurality of textiles that are structurally, compositionally and visually similar to one another. A3. The mattress assembly of claim A1, wherein the obverse face and the reverse face of the assembly are selected from a single textile or plurality of textiles that have no structural, compositional or visually similarities to one another. A4. The mattress assembly of claim 1 wherein the obverse face and the reverse face of the assembly are selected from a single textile or plurality of textiles that are structurally and compositionally similar to one another, but are of different visual appearance from one another. A5. The mattress assembly of claim A1 wherein the obverse face and the reverse face of the assembly are selected from a single textile or plurality of textiles that are structurally similar to one another, but are of different composition and visual appearance from one another. A6. The mattress assembly of claim A1 wherein the obverse face and the reverse face of the assembly are selected from a single textile or plurality of textiles that are compositionally similar to one another, but are of different structure and visual appearance from one another. A7. The mattress assembly of claim A1 wherein the obverse face and the reverse face of the assembly are selected from a single textile or plurality of textiles that are visually similar to one another, but are of different structure and composition from one another. A8. The mattress assembly of claim A1 wherein the obverse face and the reverse face of the assembly are selected from a single textile or plurality of textiles that are structurally and visually similar to one another, but are of different composition from one another. A9. The mattress assembly of claim A1 wherein the obverse face and the reverse face of the assembly are selected from a single textile or plurality of textiles that are compositionally and visually similar to one another, but are of different structure from one another. A10. The mattress assembly of claim A1 wherein the reversible nature of the ticking assembly is present throughout the entire construction of the assembly.

A11. The mattress assembly of claim 1 wherein the reversible nature of the ticking assembly is present in only a portion of the assembly. A12. The mattress assembly of claim A1 wherein said top side of said detachable and reversible mattress ticking assembly includes a plurality of textiles that are joined together with stitching, sewing, quilting, gluing or thermal bonding techniques. A13. The mattress assembly of claim A1 wherein said textiles are joined together utilizing sewing and assembly techniques that provide a retail ready appearance to end consumers in either the obverse or reverse orientation of the assembly. A14. The mattress assembly of claim A1 wherein said detachable and reversible mattress ticking assembly includes a plurality of fabric layers. A15. The mattress assembly of claim A1 wherein said detachable and reversible mattress ticking assembly includes unitarily and integrally formed textiles that possess dissimilar performance attributes on the opposing planar sides. A16. The mattress assembly of claim A1 wherein said detachable and reversible mattress ticking assembly includes a plurality of textile layers that are knitted, woven, non-woven or combinations thereof. A17. The mattress assembly of claim A1 wherein at least a portion of said detachable and reversible mattress ticking assembly includes a ticking that has cooling properties on the obverse ticking face and warming properties on the reverse ticking face. A18. The mattress assembly of claim A1 wherein at least a portion of said detachable and reversible mattress ticking assembly includes a ticking that has one predominant color on the obverse ticking face and a different predominant color on the reverse ticking face. A19. The mattress assembly of claim A1 wherein at least a portion of said detachable and reversible mattress ticking assembly includes a ticking that has one pattern or imagery on the obverse ticking face and a different pattern or imagery on the reverse ticking face.

A20. The mattress assembly of claim A1 wherein detachable and reversible mattress ticking assembly at least partially encloses said mattress core. A21. The mattress assembly of claim A1 wherein the mattress assembly includes a textile encasing that at least partially encloses the mattress core and fire barrier and that is then at least partially enclosed by the detachable and reversible mattress ticking assembly. A22. The mattress assembly of claim A1 wherein detachable and reversible mattress ticking assembly includes a zipper designed to work in reversible textile applications. A23. The mattress assembly of claim A1 wherein detachable and reversible mattress ticking assembly includes a zipper with a zipper slide that has two faces and a zipper pull on each face. A24. The mattress assembly of claim A1 wherein detachable and reversible mattress ticking assembly includes elastomeric properties. A25. The mattress assembly of claim A1 that incorporates a fire barrier element between the obverse ticking face and the reverse ticking face and utilizes a two sided, waterfall zipper flap assembly that extends the fire barrier element over the opening caused by the non-flame resistant zipper assembly. A26. The mattress assembly of claim A1 wherein said mattress includes an independent fire barrier fabric. A27. The mattress assembly of claim A1 wherein said mattress core includes foam. A28. The mattress assembly of claim A1 wherein said mattress core includes springs. A29. The mattress assembly of claim A1 wherein said mattress core includes fiber. A30. The mattress assembly of claim A1 that includes a plurality of resilient cushioning materials, selected from those including foam, springs, or fiber. A31. The mattress assembly of claim A1 wherein said detachable and reversible mattress ticking assembly covers at least thirty-percent of the outward-most surface area of the mattress assembly. A32. The mattress assembly of claim A1 wherein said detachable and reversible mattress ticking assembly covers the entire outward-most surface area of the mattress assembly. A33. The mattress assembly of claim A1 wherein said mattress core includes three or more mattress core elements having flexible foams arranged so that the IFD values of the flexible foams are sequenced so that the values on the outer surfaces are lower than the IFD values of the inner foam elements.

B1. A method for forming a mattress assembly, the method includes providing a mattress core having a top side, a bottom side, and a peripherally-extending side or sides disposed between said top side and said bottom side, positioning a detachable and reversible mattress ticking assembly over the mattress core, and wherein said mattress assembly meets the flammability standards of both 16 CFR 1632.4 and 16 CFR 1633 when tested with the detachable and reversible mattress ticking assembly disposed in each of the possible orientations, specifically with both the obverse face of the assembly and the reverse face of the assembly each being disposed outwardly toward the end user of the mattress.

It is to be understood that the above description is intended to be illustrative, and not restrictive. Numerous changes and modifications may be made herein by one of ordinary skill in the art without departing from the general spirit and scope of the disclosure as defined by the following claims and the equivalents thereof. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Also, the term “operably” in conjunction with terms such as coupled, connected, joined, sealed or the like is used herein to refer to both connections resulting from separate, distinct components being directly or indirectly coupled and components being integrally formed (i.e., one-piece, integral or monolithic). Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure. It is to be understood that not necessarily all such objects or advantages described above may be achieved in accordance with any particular embodiment. Thus, for example, those skilled in the art will recognize that the systems and techniques described herein may be embodied or carried out in a manner that achieves or optimizes one advantage or group of advantages as taught herein without necessarily achieving other objects or advantages as may be taught or suggested herein.

While the disclosure has been described in detail in connection with only a limited number of embodiments, it should be readily understood that the disclosure is not limited to such disclosed embodiments. Rather, the disclosure can be modified to incorporate any number of variations, alterations, substitutions or equivalent arrangements not heretofore described, but which are commensurate with the spirit and scope of the disclosure. Additionally, while various embodiments have been described, it is to be understood that aspects of the disclosure may include only some of the described embodiments. Accordingly, the disclosure is not to be seen as limited by the foregoing description but is only limited by the scope of the appended claims.

This written description uses examples, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A method comprising:
providing the mattress assembly comprising:
a mattress core having a top side, a bottom side, and a peripherally extending side disposed between the top side and the bottom side; and
a detachable and reversible mattress ticking assembly for at least partially enclosing the mattress core, the detachable and reversible mattress ticking assembly having an obverse face and a reverse face;
positioning the detachable and reversible mattress ticking assembly over the mattress core in a first orientation with the reverse face facing the mattress core and the obverse face facing outwardly defining a ticking surface;
removing the detachable and reversible mattress ticking assembly from the mattress core;
turning the detachable and reversible mattress ticking assembly inside out or flipping over the detachable and reversible mattress ticking assembly;
repositioning the detachable and reversible mattress ticking assembly over the mattress core in a second orientation with the obverse face facing the mattress core and the reverse face facing outwardly defining a ticking surface;
wherein the mattress assembly satisfying the flammability standards of both 16 CFR 1632.4 and 16 CFR 1633 when tested with the detachable and reversible mattress ticking assembly disposed and secured in the first orientation; and
the mattress assembly satisfying the flammability standards of both 16 CFR 1632.4 and 16 CFR 1633 when tested with the detachable and reversible mattress ticking assembly disposed and secured in the second orientation.

2. The method of claim 1 wherein:
the positioning comprises providing the obverse face in the first orientation with a first performance attribute to a customer; and
the repositioning comprises providing the reverse face in the second orientation with a second performance attribute to the customer, the second performance attribute being different than the first performance attribute.

3. The method of claim 2 wherein:
the positioning comprises providing the obverse face in the first orientation with cooling properties to a customer; and
the repositioning comprises providing the reverse face in the second orientation with warming properties to the customer.

4. The method of claim 1 wherein:
the positioning comprises providing the obverse face in the first orientation with a first visual appearance to a customer; and
the repositioning comprises providing the reverse face in the second orientation with a second visual appearance to the customer, the second visual appearance being different than the first visual appearance.

5. The method of claim 1 wherein:
the positioning comprises providing the obverse face in the first orientation with first visual appearance and a first performance attribute to a customer; and
the repositioning comprises providing the reverse face in the second orientation with a second visual appearance and a second performance attribute to the customer, the second visual appearance and second performance attribute being different than the first visual appearance and the first performance attribute.

6. The method of claim 1 wherein:
the removing comprises removing the detachable and reversible mattress ticking assembly having a soiled obverse face from the from the mattress core; and
the repositioning comprises repositioning the detachable and reversible mattress ticking assembly over the mattress core in the second orientation with the soiled obverse face facing the mattress core and the reverse face facing outwardly defining a ticking surface.

7. The method of claim 6 wherein:
the removing comprises laundering the mattress ticking assembly having the soiled obverse face; and
the repositioning comprises repositioning the detachable and reversible mattress ticking assembly over the mattress core in the second orientation with the laundered obverse face facing the mattress core and the reverse face facing outwardly defining a ticking surface.

8. The method of claim 1 wherein:
the positioning comprises positioning the detachable and reversible mattress ticking assembly in the first orientation over the top side of the mattress core, over the peripherally extending side of the mattress core, and over only a portion of the bottom side of the mattress core;
the removing comprises removing the detachable and reversible mattress ticking assembly from the portion of the top side of the mattress core, from the peripherally extending side of the mattress core, and from the bottom side of the mattress core; and
the repositioning comprises repositioning the detachable and reversible mattress ticking assembly in the second orientation over the top side of the mattress core, over the peripherally extending side of the mattress core, and over only a portion of the bottom side of the mattress core.

9. The method of claim 8 wherein the detachable and reversible mattress ticking assembly disposable over the bottom side of the mattress core comprises elastomeric properties extendable along the bottom side of the mattress core.

10. The method of claim 1 wherein:
the detachable and reversible mattress ticking assembly extends only over the top side of the core.
the positioning comprises positioning the detachable and reversible mattress ticking assembly over only the top side of the mattress core in the first orientation with the reverse face facing only the top of the mattress core and the obverse face facing outwardly defining the ticking surface;
the turning comprises flipping over the detachable and reversible mattress ticking assembly; and
the repositioning comprises repositing the detachable and reversible mattress ticking assembly over only the top side of the mattress core in a second orientation with the obverse face facing the top of the mattress core and the reverse face facing outwardly defining the ticking surface.

11. The method of claim 1 wherein the detachable and reversible mattress ticking assembly comprises a first portion extendable over a first portion of the top side of the mattress core and a second portion extendable over a second portion of the top side of the mattress core, and wherein:
the positioning comprises releasably attaching the first portion to the second portion, and positioning in the first orientation the first portion over a first portion of the top side of the mattress core and positioning a second portion extendable over a second portion of the top side of the mattress core;
the removing comprises removing the first portion from the second portion from the mattress core; and
the repositioning comprises releasably attaching the first portion in the second orientation to the second portion, and positioning the first portion over the mattress core.

12. The method of claim 1 wherein the mattress assembly further comprises a connector and wherein:
the positioning comprises closing a connector to secure the detachable and reversible mattress ticking assembly in the first orientation to the mattress core:
removing comprises opening the connector to remove the detachable and reversible mattress ticking assembly from the mattress core; and
the repositioning comprises closing the connector to secure the detachable and reversible mattress ticking assembly in the second orientation to the mattress core.

13. The method of claim 12 wherein the connector is disposed along the peripherally extending side of the mattress core between the top side and the bottom side of the core.

14. The method of claim 13 wherein:
the positioning comprises positioning a first textile flange disposed on the obverse face over the connector in the first orientation; and
the repositioning comprises positioning a second textile flange disposed on the reverse face over the connector in the second orientation.

15. The method of claim 1 wherein the mattress assembly further comprises a reversible zipper and wherein:
the positioning comprises closing a reversible zipper to secure the detachable and reversible mattress ticking assembly in the first orientation to the mattress core:
removing comprises opening the reversible zipper to remove the detachable and reversible mattress ticking assembly from the mattress core; and
the repositioning comprises closing the reversible zipper to secure the detachable and reversible mattress ticking assembly in the second orientation to the mattress core.

16. The method of claim 15 wherein:
the positioning comprises positioning a first textile flap disposed on the obverse face over the zipper in the first orientation; and
the repositioning comprises positioning a second textile flap disposed on the reverse face over the exposed zipper in the second orientation.

17. The method of claim 16 wherein:
the positioning comprises positioning a first textile flap completely over the exposed zipper and in the first orientation; and
the repositioning comprises positioning a second textile flap completely over the exposed zipper in the second orientation.

18. The method of claim 15 wherein the reversible zipper comprises a plurality of zipper chain portions, and wherein:
the positioning comprises closing one of the plurality of zipper chain portions in a first direction to secure the detachable and reversible mattress ticking assembly in the first orientation to the mattress core:
the removing comprises opening the one of the plurality of zipper chain portions to remove the detachable and reversible mattress ticking assembly from the mattress core; and
the repositioning comprises closing a different one of the plurality zipper chain portions in a different direction to secure the detachable and reversible mattress ticking assembly in the second orientation to the mattress core.

19. The method of claim 1 wherein the detachable and reversible mattress ticking assembly covers less than the entire outermost surface area of the mattress core.

20. The method of claim 1 wherein the detachable and reversible mattress ticking assembly completely covers the entire outermost surface area of the mattress core.

21. The method of claim 1 wherein the detachable and reversible mattress ticking assembly comprises at least one fire barrier layer disposed between the obverse face and the reverse face.

22. The method of claim 1 wherein:
the mattress core comprises foam.

23. The method of claim 1 wherein:
the providing the mattress assembly comprising providing the mattress assembly as an original sale into commerce.

24. The method of claim 1 wherein:
the providing the mattress assembly comprises providing a retail ready mattress assembly to consumers in either the first orientation or the second orientation of the detachable and reversible mattress ticking assembly.
